SEQUENCE LISTING (50 sequences)

SEQ ID NO: 1 - DNA, 19 bp, Artificial Sequence
  Influenza Type A Forward Amplification Primer
taaccgaggtcgaaacgta

SEQ ID NO: 2 - DNA, 16 bp, Artificial Sequence
  Influenza Type A Reverse Amplification Primer
gcacggtgagcgtgaa

SEQ ID NO: 3 - DNA, 17 bp, Artificial Sequence
  Influenza Type A Probe
tcaggccccctcaaagc

SEQ ID NO: 4 - DNA, 24 bp, Artificial Sequence
  Influenza Type B Forward Amplification Primer
ggaattgcaaaggatgtaatggaa

SEQ ID NO: 5 - DNA, 28 bp, Artificial Sequence
  Influenza Type B Reverse Amplification Primer
agaacaaattgaaagaatctgaaatggt

SEQ ID NO: 6 - DNA, 17 bp, Artificial Sequence
  Influenza Type B Probe
atgggaaattcagctct

SEQ ID NO: 7 - DNA, 23 bp, Artificial Sequence
  Influenza Subtype H1 Forward Amplification Primer
agycttcctttccagaatgtaca

SEQ ID NO: 8 - DNA, 25 bp, Artificial Sequence
  Influenza Subtype H1 Reverse Amplification Primer
agtcctgtarccatycttaattttg

SEQ ID NO: 9 - DNA, 18 bp, Artificial Sequence
  Influenza Subtype H1 Probe
tctccaaagtatgtcagg

SEQ ID NO: 10 - DNA, 22 bp, Artificial Sequence
  Influenza Subtype H3 Forward Amplification Primer
aatacgaagtgggaaaagctca

SEQ ID NO: 11 - DNA, 23 bp, Artificial Sequence
  Influenza Subtype H3 Reverse Amplification Primer
gccccrtatgtgatyctgtttac

SEQ ID NO: 12 - DNA, 19 bp, Artificial Sequence
  Influenza Subtype H3 Probe
tgagatcagatgcacccat

SEQ ID NO: 13 - DNA, 25 bp, Artificial Sequence
  Influenza Subtype H5 Forward Amplification Primer
actayccgcagtattcagaagaagc

SEQ ID NO: 14 - DNA, 21 bp, Artificial Sequence
  Influenza Subtype H5 Reverse Amplification Primer
gaccagcyaycatgattgcca

SEQ ID NO: 15 - DNA, 17 bp, Artificial Sequence
  Influenza Subtype H5 Probe
agagrggaaataagtgg

SEQ ID NO: 16 - DNA, 45 bp, Artificial Sequence
  Influenza Type A Forward Amplification Primer
gctaatacgactcactatagggagaagcaaaagcaggtagatatt

SEQ ID NO: 17 - DNA, 24 bp, Artificial Sequence
  Influenza Type A Reverse Amplification Primer
agtagaaacaaggtagttttttac

SEQ ID NO: 18 - DNA, 45 bp, Artificial Sequence
  Influenza Type B Forward Amplification Primer
gctaatacgactcactatagggagatcgctgtttggagacacaat

SEQ ID NO: 19 - DNA, 20 bp, Artificial Sequence
  Influenza Type B Reverse Amplification Primer
ctccaaaactgtttcaccca

SEQ ID NO: 20 - DNA, 43 bp, Artificial Sequence
  Influenza Type H1 Forward Amplification Primer
gctaatacgactcactatagggagaaagcaggggaaaataaaa

SEQ ID NO: 21 - DNA, 18 bp, Artificial Sequence
  Influenza Type H1 Reverse Amplification Primer
gtaatcccgttaatcgca

SEQ ID NO: 22 - DNA, 43 bp, Artificial Sequence
  Influenza Subtype H3 Forward Amplification Primer
gctaatacgactcactatagggagaactatcattgctttgagc

SEQ ID NO: 23 - DNA, 18 bp, Artificial Sequence
  Influenza Subtype H3 Reverse Amplification Primer
atggctgcttgagtgctt

SEQ ID NO: 24 - DNA, 47 bp, Artificial Sequence
  Influenza Subtype H5 Forward Amplification Primer
gctaatacgactcactatagggagatcatctgtcaaatggagaaaat

SEQ ID NO: 25 - DNA, 22 bp, Artificial Sequence
  Influenza Subtype H5 Reverse Amplification Primer
aaggatagaccagctaccatga

SEQ ID NO: 26 - DNA, 146 bp, Influenza A virus
gactacccgcagtattcagaagaagcaagattaaaaagagaggaaataagtggagtaaaattggaatcaataggaacttaccaaatactgtcaatttattcaacagttgcgagttctctagcactggcaatcatggtggctggtct

SEQ ID NO: 27 - DNA, 146 bp, Influenza A virus; identical to SEQ ID NO: 26

SEQ ID NO: 28 - DNA, 146 bp, Influenza A virus
gactacccgcagtattcagaagaagcaagattaaaaagagaggaaataagtggagtaaaattggaatcaataggaacttaccaaatactgtcaatttattcaacagtggcgagctccctagcactggcaatcatggtggctggtct

SEQ ID NO: 29 - DNA, 146 bp, Influenza A virus
gactacccgcagtattcagaagaagcaagattaaaaagagaggaaataagtggagtaaaattggaatcaataggaatttaccaaatactgtcaatttattctacagtggcgagttccctagcactggcaatcatggtagctggtct

SEQ ID NO: 30 - DNA, 146 bp, Influenza A virus
gactacccgcagtattcagaagaagcaagattaaaaagagaggaaataagtggagtaaaattggaatcaataggaacttaccaaatactgtcaatttattcaacagtggcgagttccctagcactggcaatcatggtggctggtct

SEQ ID NO: 31 - DNA, 146 bp, Influenza A virus
gactacccgcagtattcagaagaagcaagactaaacagagaggaaataagtggagtaaaattggaatcaatgggaatttaccaaatactgtcaatttattcaacagtggcgagttccctagcactggcaatcatggtagctggtct

SEQ ID NO: 32 - DNA, 146 bp, Influenza A virus
gactacccgcagtattcagaagaagcaagactaaacagagaggaaataagtggagtaaaattggaatcaatgggaacttaccaaatactgtcaatttattcaacagtggcgagttccctagcactggcaatcatggtagctggtct

SEQ ID NO: 33 - DNA, 146 bp, Influenza A virus
gactacccgcagtattcagaagaagcaagattaaaaagagaggaaataagtggagtaaaattggaatcaataggaacttaccaaatcctgtcaatttattcaacagtggcgagctccctagcactggcaatcatggtggctggtct

SEQ ID NO: 34 - DNA, 146 bp, Influenza A virus; identical to SEQ ID NO: 26

SEQ ID NO: 35 - DNA, 146 bp, Influenza A virus
gactacccgcagtattcagaagaagcaaaactaaaaagagcggaaataagtggagtaaaattggaatcaataggaatttaccaaatactgtcaatttattccacagtagcgagttccctagcactggcaatcatggtagctggtct

SEQ ID NO: 36 - DNA, 146 bp, Influenza A virus; identical to SEQ ID NO: 31

SEQ ID NO: 37 - DNA, 146 bp, Influenza A virus
gactacccgcagtattcagaagaagcaagactaaacagagaggaaataagtggagtaaaattggagtcaatgggaatttaccaaatactgtcaatttattcaacagtggcgagttccctagcactggcaatcatggtagctggtct

SEQ ID NO: 38 - DNA, 146 bp, Influenza A virus
gactacccgcagtattcagaagaagcaagattaaaaagagaggaaataagtggagtaaaattggaatcaataggaatttaccaaatactgtcaatttattcaacagtggcgagctccctagcactggcaatcatggtggctggtct

SEQ ID NO: 39 - DNA, 146 bp, Influenza A virus
gactacccgcagtattcagaagaagcaagactaaaaagagaggaaataagtggagtaaaattggaatcaataggaatttaccaaatactgtcaatttattctacagtggcgagttccctagcactggcaatcatggtagctggtct

SEQ ID NO: 40 - DNA, 146 bp, Influenza A virus; identical to SEQ ID NO: 38

SEQ ID NO: 41 - DNA, 146 bp, Influenza A virus
gactacccgcagtattcagaagaagcgagactaaaaagagaggaaataagtggagtaaaattggaatcaataggaatttaccaaatactgtcaatttattctacagtggcgagttccctagcactggcaatcatggtagctggtct

SEQ ID NO: 42 - DNA, 146 bp, Influenza A virus; identical to SEQ ID NO: 29

SEQ ID NO: 43 - DNA, 145 bp, Influenza A virus
gactacccgcagtattcagaagaagcaagactaaaaagagaggaaataagtggagtaaaattggagtcaataggaacttaccaaatactgtcaatttattctacagtggcgagttccctagcactggcaatcatggtagctggtc

SEQ ID NO: 44 - DNA, 145 bp, Influenza A virus
gactacccgcagtattcagaagaagcaagactaaaaagagaggaaataagtggagtaaaattggaatcaataggaatttaccaaatattgtcaatttattctacagtggcgagctccctagcactggcaatcatggtagctggtc

SEQ ID NO: 45 - DNA, 145 bp, Influenza A virus
gactacccgcagtattcagaagaagcaagattaaaaagagaggaaataagtggagtaaaattggaatcaataggaacttaccaaatactgtcaatttattcaacagtggcgagctccctagcactggcaatcatggtggctggtc

SEQ ID NO: 46 - DNA, 145 bp, Influenza A virus
aactatccgcagtattcagaagaagcaagattaaaaagagaggaaataagtggggtaaaattggaatcaataggaacttaccaaatactgtcaatttattcaacagtggcgagttccctagcactggcaatcatgatggctggtc

SEQ ID NO: 47 - DNA, 145 bp, Influenza A virus; identical to SEQ ID NO: 46

SEQ ID NO: 48 - DNA, 25 bp, Artificial Sequence
  H5 Primer
actayccgcagtattcagaagaagc

SEQ ID NO: 49 - DNA, 17 bp, Artificial Sequence
  influenza consensus domain
agagrggaaataagtgg

SEQ ID NO: 50 - DNA, 21 bp, Artificial Sequence
  Reverse Primer
tggcaatcatgrtrgctggtc

(19) United States
(12) Patent Application Publication
(10) Pub. No.: US 2009/0098527 A1
(43) Pub. Date: Apr. 16, 2009

(54) BIOLOGICAL ORGANISM IDENTIFICATION PRODUCT AND METHODS

(76) Inventors: Gerald W. Fischer, Bethesda, MD (US); Luke T. Daum, San Antonio, TX (US)

Correspondence Address: HAYNES AND BOONE, LLP;IP Section, 2323 Victory Avenue, Suite 700, Dallas, TX, 75219, US

(21) Appl. No.: 11/844,933

(22) Filed: Aug. 24, 2007

Related U.S. Application Data
(60) Provisional application No. 60/843,711, filed on Sep. 12, 2006.

(51) Int. Cl. C12Q 1/70; C12M 1/00; C12Q 1/68

(52) U.S. Cl. 435/5; 435/287.2; 435/6

(57) ABSTRACT

A biological organism identification product, and methods of using the same, that include a collection device to collect one or more sample organisms, a fixing and transporting composition present in an amount sufficient to kill the sample organism(s) associated with the collection device, an extraction member to extract a sufficient amount of genomic nucleic acid from the sample organism(s) to facilitate identification; and a polymerase chain reaction component into which the sufficient amount of genomic nucleic acid can be dissolved. The amplified genomic material is exposed to molecules that bind to predetermined genomic sequences, providing the identification feature of the product. The biological organism identification product may be portable, durable, and self-contained.

### FIELD OF THE INVENTION

The invention relates to a biological organism detection product and methods of using the same to: 1) rapidly identify and detect; 2) determine virulence; 3) determine drug resistance and other resistance markers from collected organisms; or a combination thereof.

### BACKGROUND

Numerous pathogens (i.e. viruses, bacteria, and parasites) cause infection and other illness in human populations worldwide. Sometimes one or more mutations in a pathogen can cause a typical illness-causing pathogen to become a full-blown pandemic. Although these pathogens and resultant illnesses are varied, one of the more prominent based on current events is the influenza virus.

The influenza virus and its variations (collectively referred to herein as “the flu virus”) are the cause for a contagious respiratory illness (commonly referred to as “influenza,” “illness,” or the “Flu”) in humans and animals (interchangeably referred to herein as a “host,” “patient,” or “subject”) that can cause mild to severe illness, and at times can lead to death. Every year in the United States alone, on average: 5% to 20% of the population gets the Flu; more than 200,000 people are hospitalized from Flu complications—and about 36,000 people die from Flu.

The flu virus spreads in respiratory droplets typically transmitted through coughing and sneezing. In human patients, the virus usually spreads from person to person, though sometimes subjects become infected by touching something with flu viruses on it and then touching their mouth or nose. Most healthy adults may be able to infect others beginning 1 day before symptoms develop and up to 5 days after becoming sick. Uncomplicated influenza illness is often characterized by an abrupt onset of constitutional and respiratory signs and symptoms, including fever, myalgia, headache, malaise, nonproductive cough, sore throat, and rhinitis.

There are three main types of influenza viruses: A, B, and C. Among type A, in particular, there are many different subtypes. The subtypes differ based upon certain proteins on the surface of the virus, specifically the hemagglutinin protein (generally referred to as “HA”) and the neuraminidase protein (generally referred to as “NA”). Presently, there are 16 known HA subtypes and 9 known NA subtypes of influenza A viruses. Many different combinations of HA and NA proteins are possible, and each combination represents a different subtype. “Human influenza virus” usually refers to those subtypes that spread widely among humans. There are three known A subtypes of influenza viruses (H1N1, H1N2, and H3N2) currently circulating among humans. Subtype H2N2, referred as the ‘Asian Flu’, was circulated within the human population from 1957-1968.

Influenza A viruses, however, are constantly changing, and are spread from birds and animals to human hosts, which leads to new influenza subtypes that can adapt over time to infect and spread more rapidly or thoroughly among humans, as has been widely reported in the mainstream press to include H5, H7, and H9 subtypes.

The H5N1 subtype, for example, has been reported as having mutated sufficiently to spread from avian hosts to human hosts. For now, the spread of H5N1 virus from human to human has been limited. Nonetheless, because all influenza viruses are constantly adapting, there is concern that an H5N1-type virus or another virulent flu subtype will be able to efficiently infect humans and spread more easily from one person to another. Additionally, because the H5N1 subtype and many other subtypes are less prevalent in, and do not commonly infect, human populations, there is little or no immune protection against them in the human population at present. It has been commonly suggested that, if H5N1 virus were to gain the capacity to spread easily from person to person, a worldwide outbreak of disease (i.e., pandemic) would likely begin.

Pandemic viruses typically emerge as a result of a process called “antigenic shift,” which causes an abrupt or sudden, major change in a virus, e.g., influenza A virus. With influenza, these changes are caused by influenza A viruses spread from birds and animals to humans, thereby creating new combinations of the HA and/or NA proteins on the surface of the virus. Such changes result in a new influenza A virus subtype. The appearance of a new influenza A virus subtype is the first step toward a pandemic. To cause a pandemic, however, the new virus subtype also would need the capacity to spread easily from person to person and be a subtype that is sufficiently dissimilar from the two typical strains (A and B) found in the human population. Once a new pandemic influenza virus emerges and spreads, it eventually becomes established and transmissible among human populations, circulating for many years as part of the seasonal epidemics of influenza.

The severity of the next pandemic cannot be predicted, but modeling studies suggest that the impact of a pandemic on the United States, and the world as a whole, could be substantial. In the absence of any control measures (vaccination or drugs), it has been estimated that in the United States a “medium-level” pandemic could cause: 89,000 to 207,000 deaths; 314,000 and 734,000 hospitalizations; 18 to 42 million outpatient visits; and another 20 to 47 million people being sick. According to the Centers for Disease Control (CDC), between 15% and 35% of the U.S. population could be affected by an influenza pandemic, and the economic impact could range between approximately $71 and $167 billion.

Biological organisms (also interchangeably referred to herein as “organisms” or “microorganisms”), such as bacteria and viruses, like influenza A, B, or a combination of organisms, and particularly pandemic influenza, threaten to quickly spread over large geographic ranges and through large populations, causing high rates of mortality and morbidity. Prior to mobilizing and implementing prevention tactics to ensure public health, it is critical to first and foremost detect and identify these organisms as soon as they appear. Early detection and surveillance to track the spread of such organisms might help mitigate the extensive damage predicted by the CDC in the event of a pandemic outbreak, e.g., influenza. Early detection is also expected to be critical in limiting or helping to treat the damage from any biological terrorism. Thus, a system to rapidly detect and identify organisms is most desirable.

Conventional techniques to detect and identify viruses, however, are not suitable for this task. Generally virus surveillance, detection and identification are time consuming (e.g., days to weeks, and in some cases, months), cumbersome to conduct, and have the potential of posing numerous health risks to health care personnel and even the general public. Conventional techniques typically require cold chain cultures and typically safety level 3 to 4 protocol, which is associated with fairly high levels of risk. The conventional surveillance, detection, and identification process (collectively referred to herein as “the surveillance process”) typically includes culturing a live target specimen (interchangeably referred to herein as “targeted specimen,” “tissue,” or “sample”), such as bird, human, or other living cells; transporting the sample to a suitable laboratory facility or other testing site, such as national, regional, or state testing laboratories; and then testing the target specimen for a range of biological organisms. Based on assays of genomic material (e.g., RNA and/or DNA) in the target sample, the organism(s) can often be identified.

Inherent in this identification and detection process is the need for bringing the target specimen back to a laboratory, thereby adding time and risk to the entire process. If the target specimen is found remotely, then it must be carefully transported to a suitable diagnostic laboratory so as to not harm, contaminate, or risk accidental exposure of the specimen—or of the people handling the specimen during transport. During transportation, for example, the specimen is typically kept in a refrigerated or near frozen condition to ensure that the specimen is kept alive and the tissues to be tested remain intact.

Thus, Applicants have discovered a need in the art for a simple to use, stable, rapid diagnostic tool and product that, rather than culturing an organism and/or sending the specimen to a remote laboratory, would allow more rapid detection and identification of biological organisms, such as microorganisms (e.g., viruses and bacteria), at or adjacent a specimen collection site. The diagnostic tool should be portable and capable of being operated remotely from a conventional laboratory, and preferably would provide safety in such an environment compared to conventional diagnostic methods used in regional facilities, such as culturing such organisms.

### SUMMARY OF THE INVENTION

The present invention meets the unmet needs of the art by providing the inventive diagnostic product (also interchangeably referred to herein as a “biological organism identification product” and a “diagnostic tool”), and methods of using the same, to rapidly detect and identify microorganisms. The diagnostic tool allows for collection of target specimens, preparation of the target specimen for assaying, isolation of genomic material, and subsequent processing of the genomic material to identify the organism. Generally, the diagnostic tool can be used in the field to collect one or more organisms and identify the collected organism(s), and provides a relatively immediate form of surveillance against potential epidemics, outbreaks, infections, and other biological organisms of interest.

Embodiments of the present invention encompass a biological organism identification product that includes a collection device to collect one or more sample organisms, a fixing and transporting composition present in an amount sufficient to kill one or more sample organisms associated with the collection device, an extraction member to extract a sufficient amount of genomic nucleic acid from one or more sample organisms to facilitate identification thereof, and a stabilized polymerase chain reaction (PCR) component into which the sufficient amount of genomic nucleic acid can be dissolved.

Preferred embodiments of the present invention include a durable, stand-alone biological organism product (referred to interchangeably herein as a “kit”) that can conduct a plurality of field diagnoses. The kit may preferably include a portable enclosure to retain the product components including the collection device, fixing and transporting composition, extraction member, and a stabilized component. The kit may also include machinery to conduct the PCR and/or a power source or power adapted to operate any machinery. In certain embodiments, the diagnostic kit also includes a plurality of active pharmaceutical ingredient doses in an amount sufficient to prevent or treat one or more conditions caused by the identified biological organism.

The present invention, in certain embodiments, relates to methods of identifying a biological organism that includes collecting a biological sample from a subject, fixing the biological sample in a sufficient amount of a fixing agent to minimize or eliminate any contamination by the biological sample, extracting a sufficient amount of genomic nucleic acid from the fixed biological sample, and assaying the sufficient amount of the genomic nucleic acid in a lyophilized polymerase chain reaction component to obtain information about the organism. In preferred embodiments, the polymerase chain reaction component has a sufficient amount of one or more primers, which identify predetermined organisms and each of which is chemically associable to a protein component specific to a biological organism. Preferably, this all occurs in a single location.

In other preferred embodiments, the method is relatively rapid compared to conventional organism detection techniques. In some embodiments of the method, no more than about 24 to 72 hours pass from the collecting of the target specimen to the assaying of the genomic material to obtain identification information. In some embodiments of the invention, the assaying is conducted for about 30 to 180 minutes, preferably 45 minutes to 150 minutes.

The invention also encompasses a reagent mix for detection of a microbial sequence, the reagent mix including one or more microbe-specific primers, probes, or enzymes, or a combination thereof, present in a mixture that is at least substantially stable at room temperature and is adapted and configured for use with a polymerase chain reaction (PCR) device. In one embodiment, the reagent mix is substantially stable at room temperature for at least about 5 days and up to two weeks. In another embodiment, the detection of the microbial sequence occurs within about 90 minutes after the microbial sequence is extracted from a sample. The reagent mix can be used to identify a microbial sequence, such as a pathogen, bacterial or viral sequence, or combination thereof. The reagent mix of the present invention, also referred to herein as a “prime mix,” can also be used to identify strains of a viral or bacterial sequence, or even sub-strains of influenza.

In another embodiment, the reagent mix can be used as part of an apparatus to facilitate determination of a microbial amino acid sequence. In preferred embodiments of the invention, the reagent mix is particularly suited to field use, and can be used in conjunction with a collection device that collects one or more biological organism samples. In additional embodiments, identification of the same occurs within about 90 minutes.

A further embodiment of the invention includes a method for detection of a microbial sequence that includes obtaining genomic nucleic acid from a biological sample and assaying the genomic material by adding the nucleic acid to the reagent mix of one or more microbe-specific primers, probes, or enzymes, or a combination thereof, wherein the mix is substantially stable at room temperature and is adapted for use with a PCR device. In another embodiment, the PCR device includes fluorescence detection equipment for real-time PCR detection.

In one preferred embodiment, the reagent mix includes an influenza strain A probe with the sequence (FAM)-tcaggccccctcaaagc, an influenza strain B probe with the sequence (FAM)-atgggaaattcagctct, an influenza subtype H1 probe with the sequence (FAM)-tctccaaagtatgtcagg, an influenza subtype H3 probe with the sequence (FAM)-tgagatcagatgcacccat, an influenza subtype H5 probe with the sequence (FAM)-agagrggaaataagtgg, or any combination thereof.

In another embodiment, the reagent mix is configured and adapted used to identify one or more sample biological organisms that have been collected by a collection device. In a preferred embodiment, the reagent mix is used to identify the one or more samples at a field site or a remote location.

In another embodiment, the reagent mix is contained in a liquid form. In a preferred embodiment, the reagent mix is present in a liquid form in a test tube, a 96-well plate, or a capillary vessel. In yet another embodiment, the mixture is lyophilized.

The invention further encompasses methods for detecting a microbial sequence which includes: obtaining genomic nucleic acid from a biological sample, and assaying the genomic nucleic acid by adding the nucleic acid to the reagent mix, wherein the mix is at least substantially stable at room temperature and is configured and adapted for use with a polymerase chain reaction (PCR) device. In another embodiment, the assaying further includes adding the reagent mix to the polymerase chain reaction (PCR) device, running the assay, and completing the assay in less than about 90 minutes. In a preferred embodiment, the assaying further includes detecting the microbial sequence in real-time using fluorescence equipment adapted for use with the PCR device. In yet another embodiment, the genomic material is from a bacteria or virus, or a pathogen. In yet a further embodiment, the genomic material is from an influenza virus.

Any of the embodiments illustrated herein stand independently, and any features or embodiments may be combined in any way, unless expressly excluded, to achieve a preferred embodiment. Additional advantages and embodiments of the invention will also become more apparent to those of ordinary skill in the art upon review of the teachings of the present application.

### BRIEF DESCRIPTION OF FIGURES

Further features and advantages of the invention can be ascertained from the following detailed description that is provided in connection with the drawing(s) described below:

FIG. 1 illustrates the stability of reagents over time at varying temperatures in accordance with one embodiment of the invention;

FIG. 2 illustrates the stability of the reagents over time at varying temperatures in accordance with one embodiment of the invention;

FIG. 3 illustrates the amplification and detection of a sequence of influenza B according to an embodiment of the present invention;

FIG. 4 includes data from an influenza H5-specific assay;

FIG. 5 is a table of primer/probe sequences used for real time RT-PCR amplification and in vitro generation of cDNA target templates;

FIG. 6 is a table of detection of influenza virus type A/B strains;

FIG. 7 is a table of detection of influenza virus types (A/B) and subtypes (H1, H3, and H5) in cultured clinical isolates by real-time RT-PCR; and

FIG. 8 is table of detection of influenza virus types (A/B) and subtypes (H1, H3, and H5) in uncultured primary clinical specimens by real-time RT-PCR.

### DETAILED DESCRIPTION OF THE PREFERRED EMBODIMENTS

The present invention provides a diagnostic tool, and methods of using the same, that permit rapid identification of one or more biological organisms of interest. Preferably, the detection and identification is sufficiently rapid so as to permit real-time or substantially real-time surveillance as to the spread of a particular organism in one or more host populations. In particular, the invention can combine recombinant DNA/RNA isolation and detection techniques to rapidly and remotely detect and identify organisms, such as microorganisms, typically pathogens. The pathogens most typically in need of identification according to the invention include microbes that cause malaria, viruses, preferably communicable viruses, and more preferably influenza, and bacteria. Advantageously, the identification can further include sub-typing and/or lineage distinction of an influenza strain or a similar species identification of another microorganism. Typically the sample is collected from a host, which provides the sample to be tested in the diagnostic product of the invention. More specifically, the present invention can advantageously allow isolation of the organism from host tissue, isolation of the genomic material of the organism, detection and identification of organisms from a target sample, on-site analysis of the organism, and identification of the organism. In some embodiments, the diagnostic tool includes a therapeutic, preventative, or prophylactic agent for administration to the host(s), which agent is selected based on the organism identified according to the invention. In other embodiments, the diagnostic tool includes detecting resistance to therapeutic agents.

The present invention provides advantages over the prior art by providing more rapid and efficient detection, classification/sub-typing, and isolation of biological organisms from a host or target specimen. In variations of the present invention, the components of the diagnostic tool can be securely enclosed in a portable enclosure to retain them in association for travel to a field site, or for use in emergency rooms and doctor's offices.

Preferred embodiments of the invention allow the identification of a biological organism at a field site. Advantageously, the enclosure contains sufficient equipment to permit multiple identifications of different collection samples in the field without requiring transport or return to a laboratory or central processing center. As used herein, the “field” encompasses any setting outside of the traditional laboratory setting. This includes emergency rooms and doctor's offices, as well as the outdoors, villages, homes, commercial offices, warehouses, streets, field hospitals, etc., and areas in which there are limited or no modern amenities (e.g., potable water and/or electricity).

In some embodiments of the present invention, the diagnostic tool includes equipment and materials to conduct and analyze the PCR for each assay. The machinery to conduct PCR is readily understood by those of ordinary skill in the art, and lower weight and bulk selections can be made according to the invention as desired to increase portability of the kit of the invention. The assays can be used in association with many types of PCR instruments, preferably with virtually every PCR instrument. Preferably, the PCR equipment is sufficiently light-weight, and adapted to draw minimal power, for increased portability and duration of use. Fluorescence-linked PCR equipment for real-time identification of a microbial sample, as is known in the art, can also be used. Although any suitable PCR equipment may be included in the product of the invention, one preferred type of PCR equipment includes the field-hardened R.A.P.I.D.® PCR equipment commercially available from Idaho Technology. Other commercially available instruments that can be used in accordance with the present invention include the Roche LightCycler®, and the ABI 7500 (7000).

In yet other embodiments of the present invention, particularly where PCR equipment is included in association with the portable enclosure, the portable enclosure includes at least one power source. While any suitable power source providing sufficiently consistent electrical output may be used, preferably the power source includes a battery, an electrical generator, a solar panel, or a combination thereof, along with any associated devices such as power cords or plug adapters to facilitate connection of the power source to any field equipment, such as a PCR device, that requires electricity to conduct the methods of the invention. In some variations of the present invention, the diagnostic product further includes replacement or repair components to maintain or enhance operation of the diagnostic tool over extended periods of time in the field without resupply. This feature is essential in some embodiments, as the product of the invention may be used in a quarantine or restricted travel environment where fresh supplies may not be available. In additional embodiments, the diagnostic tool includes any desired processor, e.g., a computer or a PDA with the relevant software, to conduct an analysis of the diagnostic testing. In certain variations, the processor determines which, if any, organisms are identified and detected. The software can be adapted and configured to search for certain likely types of organisms first depending on the field location, e.g., malaria in a tropical setting, as well as providing a knowledge base of biological organisms with information that can permit an optional, associated human operator to make any desired adjustments or to assist with the detection and identification, e.g., an analysis of the assay results.

As used herein, the term “infection,” “influenza infection,” “viral infection,” “bacterial infection,” and the like are used consistently with their accepted meanings in the art, but can also encompass the detrimental effect of a biological organism that does not result in an infection as conventionally understood. The term “methods of treating” includes methods of managing, and when used in connection with the biological organism or infection, includes the amelioration, elimination, reduction, prevention, or other relief or management from the detrimental effects of a biological organism. In a preferred embodiment, these detrimental effects include an influenza infection, influenza virus, symptoms characterizing and/or effects associated with influenza in the subject, or a combination.

In some embodiments of the present invention, the portable diagnostic is equipped with a suitable amounts of components to conduct a plurality of assays, without leaving the field or compromising sterilization or a quarantine. In some variations, the portable enclosure includes each selected component to detect and/or identify an organism in an amount sufficient to conduct at least 10 field diagnoses, preferably about 20 field diagnoses, more preferably about 50-field diagnoses, and most preferably about 100 field diagnoses. In preferred variations, the portable enclosure includes at least enough components to conduct a plurality of field analyses while maintaining the portability of the diagnostic tool. Another measure of the quantity of components present is enough of each of the types of components necessary to identify an organism over an extended period of time in the field. For example, this time period might be about 6 hours to 2 weeks, preferably 12 hours to 1 week.

In other preferred embodiments, the diagnostic tool is durable and stable, particularly during storage and transport, as well as preferably in the field during use. The components of the diagnostic tool are hardened to resist degradation even at storage temperatures of about 18° C. to 27° C., and preferably from about 20° C. to 25° C.

In addition to temperature resistance, the portable enclosure and the contents are preferably also adapted and configured to resist or prevent physical damage and/or breakage of the components therein according to the invention. The enclosure need not be complete, and may include gaps, holes, or projections to facilitate transport or storage. The product can also be arranged in modular form, such as in blister packs, so that each type of component can be stored separately or together if desired. For example, each module can hold all the components of the product, or each module can hold one type of component. Preferably, one module includes the stabilized component(s) and is stored separately under chilled conditions (i.e. less than room temperature), such as under refrigeration of less than 4° C., or more preferably under frozen conditions, until the product is ready to be transported to a remote field site. The chilled module can be combined or included with the other modular components by any conventional means to form the complete product.

Preferably, it is a complete enclosure sufficient to resist water penetration, and the enclosure is preferably waterproof. In some variations, the portable enclosure optionally includes an apparatus to facilitate portability, such as a handle, a strap, etc. In other variations, the portable enclosure includes a fastenable and resealable opening and closing feature that allows access to the components, safe storage of the portable enclosure, and/or maintenance of sterilized components, e.g., one or more door or hatch handles, zippers, latches, or the like.

The enclosure itself may be made of any sufficiently resilient packaging material available to those of ordinary skill in the art that can protect fragile contents such as glassware or PCR equipment or otherwise help increase the integrity of the components of the product, particularly any lyophilized reagents or samples that are present. Preferably, the enclosure and components are made of a suitable non-breakable material other than glass to facilitate shipment or transport of the enclosure to a field site. Examples of such packaging material that can be included in forming the portable enclosure include: aluminum or plastic foil, blister packs, cardboard or other paperboard, or a polymeric or other plastic component such as a thermoplastic polyolefin, or a temperature-stable polymer. For example, the resilient packaging material may include a temperature-stable polymer such as a propylene homopolymer or a copolymer of at least 50 mole percent of propylene and at least one other C₂ to C₂₀ alpha-olefin, or mixtures thereof. Exemplary alpha-olefins of such copolymers include ethylene, 1-butene, 1-pentene, 1-hexene, methyl-1-butenes, methyl-1-pentenes, 1-octene and 1-decene, or a combination thereof.

The enclosure may be formed through any available process, which may be selected by one of ordinary skill in the art with reference to the type of material. For example, a polymeric material may be molded or extruded. While any shape may be imparted to the enclosure that is sufficient to enclose the components, preferably the enclosure has a base that is sufficiently stable (e.g., flat) that it will not tip over during storage or use. The enclosure can be arranged to fold open to a table if desired, with the legs folded up inside when closed and an outer surface of the enclosure forming the tabletop surface when opened. The contents may be placed on the opened table to provide a convenient workbench when conducting the methods of the invention. Other convenient arrangements of the enclosure can be envisioned, such as an opening to form a shelf that can be placed on an existing table.

The diagnostic product includes one or more types of collection device to capture, collect, or otherwise take the target specimen from the host, and then contain or hold the specimen for further analysis. The specimen may include tissue, blood, saliva, or another biological product testable for genomic material from microorganisms present in the specimen. Any suitable collection device may be used to accomplish this goal. For example, swabs may be used to collect mucosal samples. Samples are preferably collected from the skin, nasal passages, oral passages, or a combination thereof. If necessary, blood may be drawn to obtain the necessary sample. Preferably, the collection device is sterile. Other preferred collection devices are those compatible for analysis with processing machinery, assay compositions and volumes, and/or size requirements for portability or as provided by the diagnostic kit described herein. Sufficient numbers of collection device, as further discussed herein, should be included to permit use of the diagnostic product in the field for an extended period of time in the event of a crisis.

In a preferred embodiment, the target specimen, or tissues or cells thereof, are immediately preserved upon, or shortly after, collection. Preferably, the target specimen is treated to kill the biological organism(s) contained therein. A preferred embodiment of the invention includes a fixing and transporting composition, which is typically a liquid and preferably a solution, emulsion, or suspension. The fixing and transporting composition helps minimize or eliminate contamination of the sample or the environment, as well as inhibiting or preventing escape of the sample. Preferably, the fixing and transporting composition includes alcohol (e.g., ethanol), sodium cyothianate, guanidine thiocyanate, or a combination thereof. Any suitable fixing and transporting composition (also referred to herein as a “fixing and transporting agent”), may be used to kill (i.e., fix) the organisms by disrupting a cellular membrane in the organism. The specimen may then be more safely transported to the assay site, which can be across a room from a patient, in a nearby room, or even more remote such as across the street or in a different part of the field site. For example, collection of genomic material from patients may occur in one tent or room, while the assays and PCR equipment are located nearby, such as within a few minutes drive. The collection device may be dried after being exposed to the fixing and transporting composition, but preferably the collector remains in the composition until just prior to the assay.

The collection and fixing of the target specimen may be arranged as follows. A cotton-tipped swab can be contacted with the nasal passages of a host. The organism(s) collected are then fixed. One fixing step that can be carried out in accordance with the present invention is generally described in Krafft, A. E., et al., Evaluation of PCR Testing of Ethanol-Fixed Nasal Swab Specimens as Augmented Surveillance Strategy for Influenza Virus and Adenovirus Identification, Journal of Clinical Microbiology, April 2005, Vol. 43, No. 4, pages 1768-1775, which is incorporated herein by express reference thereto. Another method of accomplishing the fixing step is suggested in Chomczynski and Sacchi (1987, Anal. Biochem 162: 156-159), through the use guanidine thiocynate.

The swab is either soaked in or placed in alcohol to kill the organism cells while sufficiently preserving the specimen for analysis. As used herein, “preserve” means that the nucleic acid material of the organism is not unduly damaged in the fixing process so that an assay and identification can be conducted. The fixing results in a significantly safer specimen that does not require cold-temperatures for preservation, such that the non-living specimen can be transported and even shipped, via standard postal mail if necessary. Further, because the specimen is killed, there is no risk of further outbreak or infection in the carrier or those associated with shipment of the sample if necessary. For example, even though the entire method can be performed in the field, it may be desired to conduct the assay and identification in a laboratory, either in the first instance or as a second trial to confirm the results of the field identification. In a preferred embodiment, the fixing and transport composition encompasses a non-hazardous, fixed specimen that can be processed using the components of the diagnostic tool.

Moreover, a second collection device can be used and stored differently from the fixing and transporting composition. For example, a second swab could also be included in the collection device and used to collect a sample from a host for a second assay or a different type of assay, such as in a regional laboratory or on different equipment, to help confirm the diagnosis later. For example, one swab can be used to collect genomic material and assay the organism at the field site, while a second swab can collect genomic material and be disposed in a chilled package, such as a refrigeration or freezer unit for up to about 4 days, preferably capable of being transported to a remote laboratory to further analysis. The second swab can be used to help identify organisms and to test for new vaccine candidates. One example of portable, cold storage suitable for use with the invention is the American Thermal Wizard, available through American Thermal Wizard International.

The extraction member is used to extract genomic material, or other relevant biological material, to characterize and identify one or more organisms from the target specimen. As used herein, the “genomic material” includes nucleic acids, such as RNA and/or DNA, that provide information as would be known to one of ordinary skill in the art to facilitate identifying and characterizing an organism of interest. Buffers, centrifuges, syringes, etc., as would be known to one skilled in the art, are exemplary extraction members suitable for the present invention. Suitable extraction techniques include those generally described in Matthews, C. K., et al, Biochemistry, Second Edition, The Benjamin Cummings Publishing Co., 1996 and Tortora, G. J., et al., Microbiology: An Introduction, The Benjamin Cummings Publishing Co., 1992, which are incorporated herein by express reference thereto. Generally, the extracted genomic nucleic acid is present in an amount from about 0.1 microliters to about 10,000 microliters, more preferably from about 1 microliter to about 1000 microliters, and more preferably from about 10 microliters to 100 microliters. An exemplary amount of nucleic acid is 25 microliters.

With respect to the extraction member, and other devices and/or apparatus in the diagnostic tool, it is preferable to maintain the equipment and identification environment in sterile or uncontaminated form. The diagnostic tool may optionally, but preferably, include one or more components to sterilize or maintain sterilization as would be known to one skilled in the art in certain embodiments. The fixing agent may also be selected to provide suitable sterilization, which may be a desirable way to reduce the number of different optional components necessary to function effectively in the field.

In the present invention, the PCR component when preferably included in the product is preferably suitable for portability and field use and analysis. One exemplary PCR assay includes real time reverse transcriptase-PCR (rRT-PCR), as generally described in Das, A., et al., Development of an Internal Positive Control for Rapid Diagnosis of Avian Influenza Virus Infections by Real-Time Reverse transcriptase-PCR with Lyophilized Reagents, Journal of Clinical Microbiology, September 2006, Vol. 44, No. 9, pages 3065-3073, which is incorporated by reference herein. An exemplary protocol for conducting the rRT-PCR is also included in the Das et al. publication.

In preferred embodiments, the pre-selected PCR reagents are premixed to include target primers and probes in one or more PCR-adapted vessels. In the most preferred embodiments, the vessels are adapted and configured to be compatible, operable and functional with the selected PCR machinery (i.e., the PCR device). For example, capillary pipettes that are sized and dimensioned to be operatively associated with the included PCR device may be directly inserted in the compatible PCR equipment for rapid use. In accordance with certain embodiments of the invention, these contain stabilized wet reagents adapted and configured for use with the genomic material in a PCR device. In one preferred embodiment, all of the PCR reagents are stabilized. The stabilized reagents can be already disposed in a PCR holding device to which a liquid including the extracted genomic material is later added. Alternatively, PCR-usable vessels may include stabilized materials or spherules that can universally fit various PCR machinery or stabilized PCR materials from the diagnostic product can be put into solution or other liquid containing the extracted genomic material. The solution is then added to the PCR holding device, which can then be placed in the PCR machinery. In one example, the PCR holding device contains the stabilized materials and the extracted genomic material in solution is added to it. Or, by way of another example, stabilized material can be added to a cuvette to which is added extracted genomic material in solution, or vice versa, and then the proper amount of that solution can be added into the PCR holding device (e.g., a pipette), and placed in the PCR machinery.

In another preferred embodiment of the invention, the desired PCR components used to contain and assay selected types of samples are pre-loaded into one or more vessels and are then stabilized to maintain the quality of the vessel and its contents for field use once the extracted genomic material is combined.

The PCR assay is preferably included in the product for field use, and encompasses detecting the genomic material of the organism. Accordingly, in preferred embodiments, at least one reagent of the PCR component includes one or more primers and/or one or more probes specific to the detection of one or more predetermined biological organisms. The diagnostic tool preferably includes primers predetermined and preselected for use in identifying certain specific organisms. As used herein, the primer is the composition used to detect specific genomic material, such as forward and reverse primers, and a probe is a sequence that binds to a microbial sequence for amplification. The PCR provides amplified genomic material, which can chemically associate with certain primers and/or probes. In some embodiments, the primer, probe, or combination can include an anti-sense nucleic acid sequence that is chemically associable with the genetic material of a detected organism. In other embodiments, the primer, probe, or combination is chemically associable to a protein or component specific to the extracted genomic material from the biological organism. This specificity facilitates rapid identification of the organism, and/or sub-typing, e.g., to include influenza A subtypes H1, H3, H5, H7, H9, as may be readily determined by those of ordinary skill in the art particularly with reference to the present disclosure. For example, if a primer and/or probe specific to H5N1 influenza chemically associates in the assay, then there is evidence of the detection and identification of, e.g., H5 influenza in the targeted specimen.

The types of primers, probes, or both included in the diagnostic tool are preferably pre-selected by one of ordinary skill in the art. In some variations, a wide variety of primers, probes, or a combination thereof are included to detect and identify any of a corresponding wide variety of organisms, particularly where there is no advance knowledge of the type of organisms expected when using the diagnostic product. In situations in which a particular organism is suspected (e.g., H5N1 influenza), the range of primers, probes, or a combination thereof that are loaded into the portable enclosure may be focused on H5N1 influenza and influenza with a similar genomic composition, or may be exclusively those used for influenza. For detection of SARS, the primer, probe, or combination can be a sequence that is specific to a SARS virus. Additionally, the primer, probe, or combination can be specific to a sequence for a strain or substrain of SARS. The PCR components may include a redundancy of primers and/or probes to ensure detection of a suspected organism and genome thereof. The library of primers and probes is generally increasing as the genomic sequence of new organisms are mapped, which can permit more suitable primer and probe selection for future uses of the diagnostic product. Descriptions of certain maps, primers, and probes that may be useful in connection with the invention includes those described in the Das publication, which is incorporated herein by express reference thereto. Preferably, the influenza primers and probes are designed to detect at least one strain of influenza encompassing the A or B types, and more preferably each of the sixteen H subtypes and each of the two B lineages.

In a particularly preferred embodiment, the PCR component is a reagent that includes one or more of microbial primers, probes and enzymes, or any combination thereof, present in a mixture. This mixture may further include standard PCR components such as water, buffer, nucleotides, polymerase, or the like, or any combination thereof. The mix of standard components is known in the art as a master mix. One or more microbe-specific primers, probes, enzymes, or any combination can be added to the master mix to create the prime mix. The prime mix can have one, two, three or four or more microbial primers, probes and/or enzymes. The microbial primers, probes and/or enzymes can be specific to infective viral or bacterial agents in general, or to specific agents, such as those associated with influenza, dengue fever, malaria, HIV, SARS, MRSA, and tuberculosis. In one preferred embodiment, the primers, probes and/or enzymes in the prime mix are specific to the influenza strains A, B, or both. In another embodiment, the primers, probes, and/or enzymes are specific to sub-strains of influenza, such as H1, H3, H5, H7, and H9. In a further embodiment, the primers, probes, and/or enzymes are inclusive for all substrains of influenza (H1-H16 and N1-N9) and the two primary flu B circulating strains.

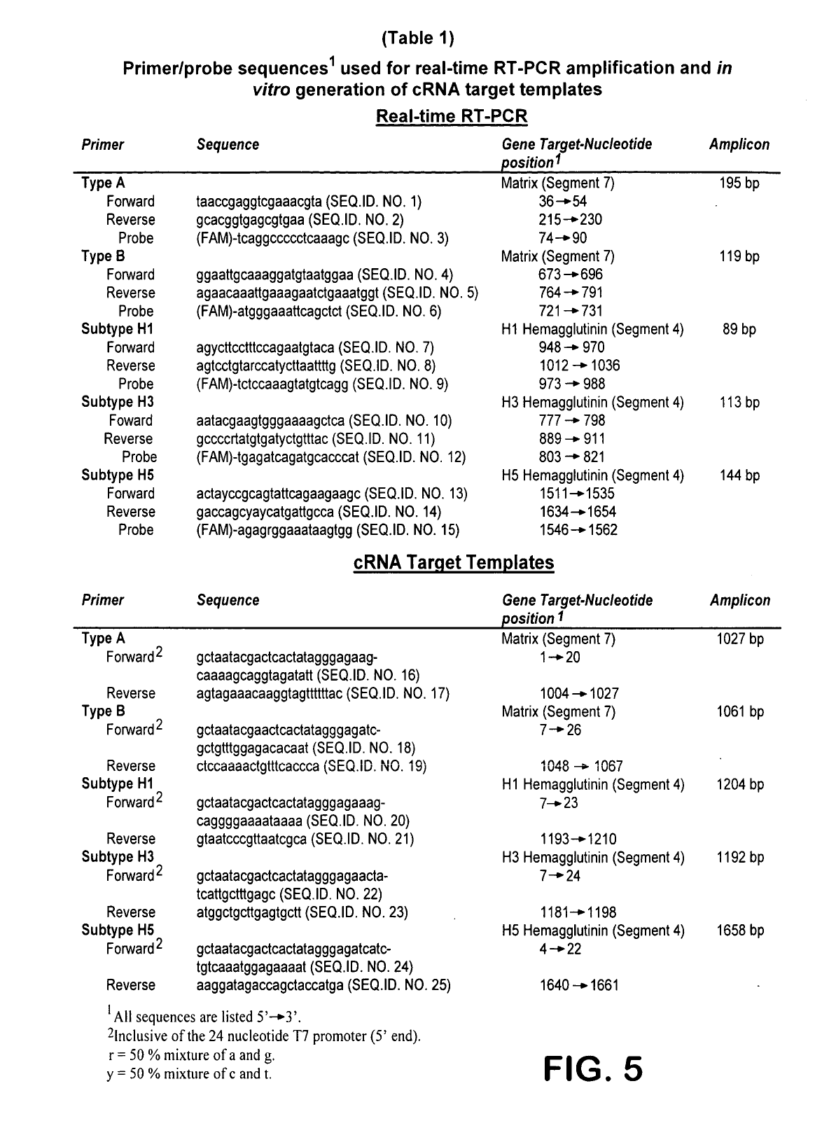

By way of example, certain primers and probes specific to influenza strains or types, or sub-strains or sub-types, are presented in FIG. 5 as well-suited to the present invention. The probes of FIG. 5 are oligonucleotide sequences located internal to the forward and reverse amplification primers. These oligonucleotides are dual labeled, containing one of several types of 5′ fluorescent reporters, e.g., 6-Carboxyfluorescein N-succinimidyl ester (FAM) and one of several types of 3′ quenchers, e.g., TAMRA, MGB Dark Quencher, etc. The sequences for influenza strains A and B are located on RNA Segment 7, which includes the open reading frames of the two matrix genes, M1 and M2, that are highly conserved among influenza virus strains. The sequences for influenza sub-types are located on RNA Segment 4, which codes from the hemagglutin (HA) protein. Nucleotides “Y” and “R” are degenerative nucleotides that have been included in sequence positions that exhibit high variability, and represent mixtures of nucleotides “C and T” and “A and G”, respectively. Degenerative bases are used when there is genetic variability among strains at a particular nucleotide position within the genome.

The PCR component, which can be any component as discussed herein or can be, or can include, the prime mix, is present in an amount to sufficiently dissolve the extracted genomic nucleic acid material. Where the component is lyophilized, it may be necessary to reconstitute the material with added water or other suitable solvent before, with, or after combining the extracted, fixed, genomic material. The PCR vessel loaded with the genomic material is placed in the PCR machine for a prescribed period of time. For example, the assay time may take from about 30 minutes to 180 minutes, preferably about 45 minutes to 150 minutes. In a more preferred embodiment, the assay time is about 60 minutes to 120 minutes. In embodiments where the PCR component is the prime mix, detection preferably can be achieved within approximately 90 minutes from extraction. These times are intended to encompass preferred times for both DNA amplification, as well as RNA amplification that includes about 30-35 minutes for the reverse transcriptase step to convert the RNA to DNA. The exact time may be readily determined by those of ordinary skill in the art depending upon the material to be assayed and the type of PCR device selected.

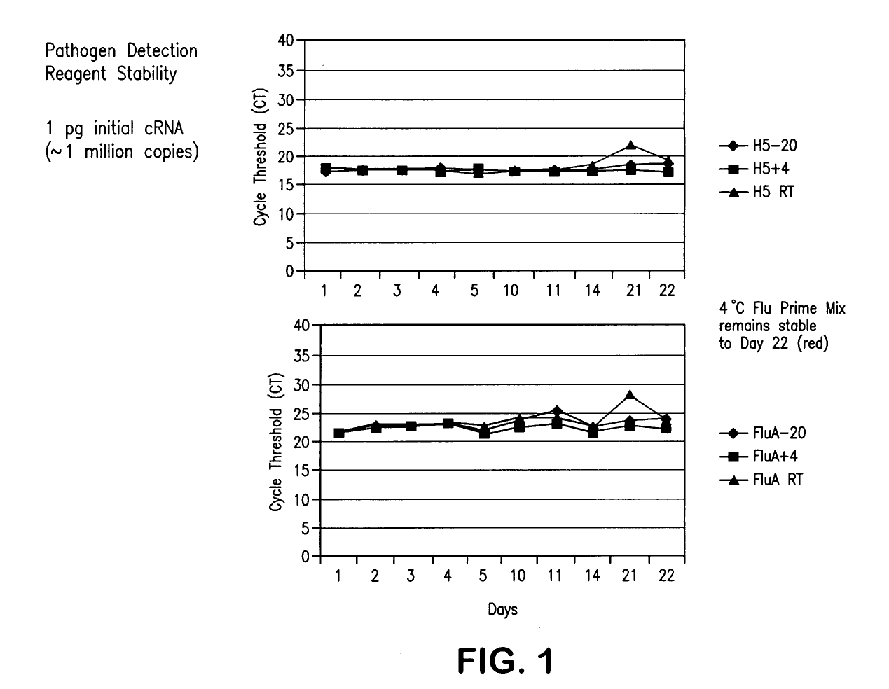

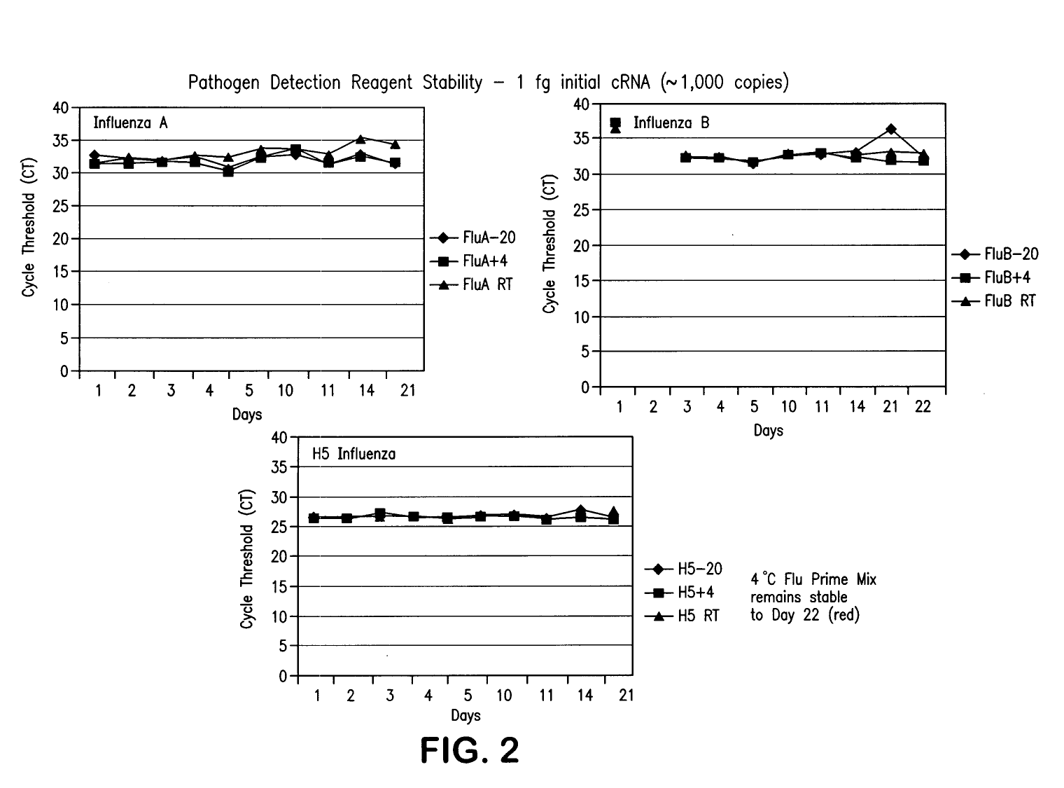

The PCR reagents used in accordance with the present invention, in preferred embodiments, are designed to be at least substantially stable, and more preferably, stable. Specifically, the reagents in the form a prime mix of the present invention, are preferably substantially stable at room temperature, and this stability is measured and standardized as shown in FIGS. 1 and 2. FIG. 1 illustrates the stability of the pathogen detection reagents. The standard chosen here is 1 picogram of initial cRNA from Flu A and H5 influenza. The Flu A and H5 samples were stored at −20° C., 4° C., and at room temperature (about 25° C.). Along the Y axis is the number of PCR cycles that have been run to register a reading. Stability as defined by the present invention is functional stability of the prime mix, which is indicated by detection of the sequence through amplication and fluorescence. In FIG. 1, functional stability is indicated by detection of the sequence (with initial sample containing 1 pg of cRNA) up to 35 PCR cycles. The level of baseline fluorescence signifying a positive reading of a sample, and thus detection thereof, is referred to at the C_{T} value. The Cycle Threshold (C_{T}) is defined as the fractional cycle number at which the fluorescence passes the threshold. The threshold level is the Delta Rn used for C_{T} determination in real-time assays. The level is set to be above the baseline and sufficiently low so that it is within the exponential growth region of the amplification curve. The Delta Rn is the magnitude of the signal generated by the specified set of PCR conditions (Delta Rn=Rn−baseline). (See ABI Relative Quantification Users Guide for 7300/7500/7500 Fast Systems, Copyright 07.2006.)

As shown in FIG. 1, stability is defined as the ability to reach C_{T} at 35 cycles or less, using 1 pg of cRNA in the starting sample. Thus, “substantially stable” encompasses prime mixes of the invention that are detectable at 1 pg through no more than 35 PCR cycles after the prime mix is stored at the specified temperature over time. For example, “substantially stable” includes prime mixes stored at room temperature for up to about 2 weeks, preferably up to about 4 weeks, and more preferably up to about 2 months or even about 3 months, where the prime mix is still useful and functioning for its intended purpose as measured by detection at 1 pg amounts in no more than 35 PCR cycles. The prime mixes are at least substantially stable for even longer periods of time at the various tested temperatures below room temperature.

FIG. 2 is a Pathogen Detection Reagent Stability Study, wherein the initial amount of cRNA is 1 femtogram. Here the cDNA is from Flu A, Flu B, and H5 influenza. FIGS. 1 and 2 illustrate the superior stability of a selection of prime mix reagents of the present invention at all three temperatures. As can be seen from the graph, the prime mix reagents for all three viruses remains stable at −20° C. and 4° C. to day 22. The stability profile at room temperature is also surprising, lasting about two weeks or more. It is further noted that the deviation around day 21 in the FIGS. 1 and 2 is likely due to the fact that the sample size was extremely small. As can be observed from the graph, the results of testing on day 22 showed a return to the detection level consistent with earlier testing days, indicating that stability beyond day 22 is reasonably be expected.

Based upon the data presented in FIGS. 1 and 2, substantial stability is achieved for extended period of time when the sample is kept frozen at about −20 to about 0° C., when the sample is stored above freezing such as in a refrigerator from above 0° C. to about 4° C., and when the sample is stored above refrigeration temperature to room temperature such as in the range of about 5° C. to about 27° C. In another embodiment, substantial stability can occur even at temperatures from above about 27° C. to 40° C. or about 27° C. to 50° C. Without being bound by theory, it is believed that the colder to temperature, the longer the sample will remain stable. For instance, the sample may be stable when frozen for 3-6 months, when refrigerated for 1-4 months, and at room temperature for about one month.

Stability may preferably be achieved by a variety of means as in known in art. such as by lyophilizing the components. In one embodiment, stability may be enhanced by maintaining the lyophilized components of the invention below room temperature until the product is ready to be assembled, transported, or used. Based on the data from the stability studies as discussed above, stability can be achieved and maintained over a period of at least two months, more preferably three months.

This stability can be achieved by the lyophilization of all the PCR reagents before being loaded in the vessels included in the enclosure or before being packaged (e.g., foil or blister packs) in association with vessels. Lyophilization procedures and reagents are described in Das, A, et al., Development of an Internal Positive Control for Rapid Diagnosis of Avian Influenza Virus Infections by Real-Time Reverse transcriptase-PCR with Lyophilized Reagents, as described above. It is noted, however, that the lyophilization of the reagents in the Das paper includes lyophilization of a master mix (nucleotides, buffer, polymerase) only.

In accordance with one embodiment the present invention, the lyophilized reagents are disposed in the PCR-adapted vessels. Advantageously, including and using lyophilization for all PCR reagents can achieve one or more of the following: minimize efforts to assay and otherwise detect and identify organisms, particularly in difficult environments or under difficult conditions, increase assay reliability due to minimized or avoided degradation of the components, require less skilled operators of the diagnostic tool of the invention, and ensure greater durability of all components.

Stability may be achieved through any suitable method known by those of ordinary skill in the art. While lyophilization is one preferred embodiment to prepare the prime mix of the invention, the reagents can be encapsulated, e.g., in a liposome or paraffin bead, that dissociates in a PCR at typical operating temperatures, as will be readily determinable by those of ordinary skill in the art. As the PCR process is typically run at about 50° C., the liposome or bead can be designed to melt or dissolve at this temperature. Stability can also be achieved by providing all the components of the prime mix in liquid form in either a test tube, a 96-well plate, or a capillary vessel of plastic or glass. Those of ordinary skill in the art will envision other available methods to achieve the necessary stability of the prime mix of the invention based on the guidance provided herein.

Following the assaying of the genomic nucleic acid with the PCR instrument, primers, and/or probes, the results can be analyzed. Whether one or more primers and/or probes have associated with one or more organisms is generally known to those of ordinary skill in the art, based on chemical indicators, colors, and other observable results based on the reactions. For example, the association between the primers and/or probes with genetic material from the organism may be detected by fluorescence to facilitate detection of the biological organism. In certain embodiments of the present invention, the kit includes an analyzer to provide diagnostic information as to which probes, if any, provide positive results (e.g., a positive result is detection by of an organism by a probe specific for that organism). In some embodiments, the analyzer is incorporated with the PCR assay.

Following assay and identification, the diagnostic product of the invention optionally but preferably also includes the necessary pharmaceutical agent, along with pharmaceutically acceptable carrier, in the field to treat the disease or condition associated with the organism, or one or more symptoms associated therewith. Although such pharmaceutical compositions can be packaged with the product, preferably they are packaged separately from the product, particularly where the conventional pharmaceutical components are less stable than the lyophilized components. This permits pharmaceutical compositions with a longer shelf life to be included in the portable enclosure or with other modular components thereof just prior to use or transport to a field site or nearby storage site. Preferably, the diagnostic product includes a plurality of desired pharmaceutical compositions in doses in a number sufficient to prevent or treat one or more conditions caused by a selection of biological organisms depending on which organism is detected and identified. Preferably, these are formulated conventionally in a desired dosage form and strength, such as a tablet, capsule, patch, solution, lotion, or the like. Varying dosage strengths may be provided for certain types of pathogens, as appropriate. Indeed, the portable enclosure can be loaded with different components of any kind, such as active pharmaceutical ingredients, depending on the expected biological organisms or patient population one of ordinary skill in the art might encounter, based, for example, on field location, first responder reports, prior experience, or the like. If the patients are expected to be pediatric or geriatric, a larger portion of liquid formulations may be selected, by way of example.

The active pharmaceutical ingredient optionally, but preferably associated with the portable enclosure may include one or more vaccines, biologics, therapies, drugs, prophylactics, compositions (e.g., immunogenic), antidotes, treatments, cures, or any other medical item that is directed towards the treating or preventing of selected biological organisms. For example, if certain influenza strains are identified, the diagnostic tool may include Tamiflu® (Roche Pharmaceuticals Inc., New Jersey, USA) to treat the corresponding influenza infection. If certain bacteria are identified, the diagnostic tool may include certain antibiotics, such as azithromycin, effective in treating the corresponding bacterial infection. The dosages will, in any case, be present in a therapeutically or prophylactically effective amount.

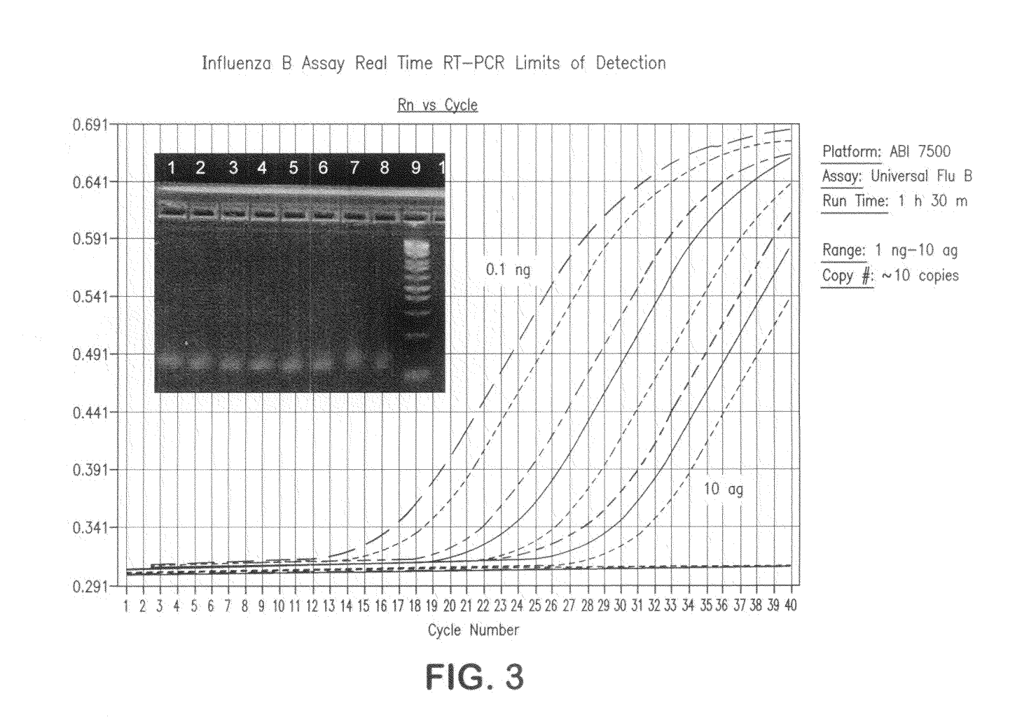

In a prototype assay according to one embodiment of the present invention and where the microbe is a virus, RNA is extracted from a swab or tissue and added to the prime mix. Optionally, a control can be added to the mix. The control can be a positive control such as cRNA of the target microbe to allow a comparison to determine the identity of the sample, and/or a negative control, such as RNase-free water. The control(s) can also be run in a separate assay, either concurrently or sequentially. Next, the prime mix with the added RNA sequence to be identified is run on any suitable PCR instrument based on the guidance herein coupled with that known to those of ordinary skill in the art. Detection occurs within about 90 minutes from the time of extraction of the sample. In an assay using the prime mix, a very small number of copies of the microbial sequence are needed for identification to occur. FIG. 3 is a standard curve of concentrations from about 0.1 ng to 10 ag showing fluorescence readings of a sample having only 10 copies of a sequence in the 10 ag concentration. The gel in the upper left hand corner of FIG. 3 was run to verify the data shown fluorescence curves. In FIG. 3, influenza B is the sample being identified. As shown by the curve, 10 copies is sufficient for identification. It is further noted that, in accordance with the present invention, it is possible to identify a sequence with 5 or fewer copies of the sequence.

Methods of the present invention include detection of a microbial sequence including obtaining genomic material from a biological sample and assaying the genomic material (i.e., nucleic acid) by adding the sample material to the prime mix. In certain embodiments of the invention, the components of the prime mix may be present in liquid form in one or more laboratory vessels, such as a test tube, a 96-well plate, or a capillary of plastic or glass. The assay is preferably adapted for use with any PCR instrument, which may also include any fluorescence instrument commercially available or known in the art, for real time detection of the microbial sequence.

The prime mix of the present invention can also be used for disease surveillance. Disease susceptibility diagnostic assays can be augmentative genetic prime mix assays of the invention that provide additional medical information about one or more bacterial or viral pathogens, such as resistance markers resulting from known genetic polymorphisms/mutations that confer resistance to known drug treatment modalities.

For example, a growing number of influenza A (H3N2) isolates obtained from patients in the U.S. revealed that 92.3% contain a change at amino acid 31 (S31N) in the M2 gene known to be correlated with adamantane resistance (e.g., amantadine and rimantadine) and 2 of 8 influenza A (H1N1) strains contained the same mutation (JAMA, Bright et al, 2006). Adamantane resistance among influenza A (H3N2) and some H1N1 strains is highlights the clinical importance of having rapid (point of care) surveillance for antiviral resistance. In accordance with one embodiment of the invention, the prime mix can target resistance markers (e.g., by use of a probe) for neuraminidase inhibitors.

Identification of influenza to a specific strain and sub-strain can be determined by the following. It should be understood that any viral or bacterial agent may be targeted according to the invention, and that most references herein to influenza may be replaced with any other suitable viral or bacterial agent. First, an assay is run by the method outlined above to determine if an influenza pathogen is present in a sample. If an influenza infective is present in the sample, a second test can be run to determine if the virus is influenza strain A or B. If the organism is identified as influenza A, one or more additional tests can be performed to determine the subtypes, such as H1, H3, H5, etc. Preferably, this is all achieved using the same sample so that no additional samples are required from the patient or subject. Alternatively, a single test can be run with the patient sample and the primers, probes, enzymes, or any combination for multiple strains and subtypes.

There are several advantages that can be imparted by use of the prime mix of the present invention. First, when the prime mix is already assembled in a suitable container for insertion into a PCR device, it is unnecessary to store multiple individual reagents and excess laboratory equipment at a site or to carry it in the portable enclosure of the invention. This is particularly beneficial in areas where access to storage, refrigeration, transport, or any combination, is not readily available, and where mobility is desired (as it is no longer necessary to transport and store individual reagents). A further benefit of a pre-assembled prime mix, when used, is that the identification process is streamlined, requiring less time, less mixing and pipetting, less cleaning or recycling of containers, and less margin for error in measuring. The simplified process can reduce the opportunity for user error and contamination, and can advantageously expedite assay results.

Additionally, the prime mix can permit easy and rapid detection of a sample microbial sequence. Current detection methods for pathogens and disease resistance can take up to several days. In contrast, by using the prime mix of the present invention, detection can be achieved within about 90 minutes in one embodiment. As illustrated by FIGS. 1-3, detection can be achieved using any standard PCR instrument and a small amount of the starting sequence. With a starting amount of 1 million copies of a sequence, detection can be achieved in fewer than 35 cycles. Improved detection allows identification of smaller samples. This is particularly advantageous in instances where it is desired to run multiple tests from a single sample, leaving more sample available for additional tests. This feature may also be beneficial in the event that a sample has been compromised, perhaps through transit or exposure, and less sample is intact for testing.

Surprisingly, the prime mix exhibits markedly increased stability, lasting several days or longer at room temperature. In another more preferred embodiment, the prime mix exhibits substantial stability for at least about two weeks, and up to about one month at room temperature. Stability for an extended period of time at room temperature (approximately 23° C.-27° C.) imparts great flexibility in use in both traditional and non-traditional environments. For example, the assay can be used more reliably in hospitals, doctor's offices, as well as in remote locations, such as in areas that have been subject to a bioterrorist attack, natural disaster, or at battlefield. The invention can be used at airports and border crossings to help minimize or prevent infected individuals from spreading disease.

Further, the prime mix can provide great flexibility and compatibility of use. The prime mix is adapted for use with several rRt PCR formats, and is available ready-to-use in reaction vessels for direct and immediate analysis. The prime mix can be used with any extraction or purification kit, and can include any standard master mix components (buffer, nucleotides, polymerase) available in the art as well as components described herein.

It is noted that the present invention encompasses the prime mix alone, existing independently from any apparatus or kit. Separate, additional embodiments of the present invention are directed to the use of the prime mix with other apparatuses, kits, etc.

As used herein, “an effective amount” would be understood by those of ordinary skill in the art to provide a therapeutic, prophylactic, or otherwise beneficial effect against the organism, its infection, or the symptoms of the organism or its infection, or any combination thereof.

As used herein, the term “rapid” encompasses a period of time that is shorter than the time involved for the conventional method of detection and identification, which typically involve culturing the target specimen, shipping it to the laboratory (usually in a cold chain), assaying the extracted nucleic acid, and then analyzing the results of the assay. In certain embodiments, the time to use the components of the diagnostic tools, i.e., perform the methods from beginning the collecting through the assaying, is less than about 24 hours, more preferably less than about 12 hours, even more preferably less than about 6 hours, and yet more preferably less than about 3 hours. In preferred embodiments, the assaying step is fully conducted within about 30 minutes to 3 hours, preferably about 45 minutes to 150 minutes, or more preferably about 60 minutes to 2 hours of the collecting. In one preferred embodiment, the assaying is completed within about 5 to 150 minutes, preferably about 10 to 120 minutes, of the collecting.

In another embodiment, the assaying is conducted within about 1 to 120 minutes, preferably about 5 to 90 minutes, of the collecting. The identifying step can occur, in certain preferred embodiments, within the same time frame from the collecting as those set forth above for the assaying.

As used herein, the term “efficient” encompasses a comparison between the diagnostic tool and methods of the present invention compared to the tools and methods in the art, which require one or more additional steps including ensuring the viability of the culture sample, shipping the sample to a remote location away from the collection site, or both. The conventional methods typically require transport to a laboratory facility of sufficient security to handle hazardous pathogens, while the invention safely moves the detection assay into the field for rapid detection and surveillance.

As used herein, the “target specimen” is a host of the organism from which the biological sample is collected for detection, identification, or both. As used herein, the term “patient” (interchangeably referred to herein as “host,” or “subject”) refers to any host that can be infected with an organism, such as influenza. Preferably, the host includes any avian as well as any mammal. In one embodiment, it includes any avian host. In a preferred embodiment, patient includes any mammalian host, such as humans, whales, gorillas, dogs, cats, cattle, horses, pigs, livestock, and poultry, etc. In a more preferred embodiment, the host is a human host.

As used herein, the term “kit” may be used to describe variations of the portable, self-contained enclosure that includes at least one set of components to conduct the methods of the invention including collecting a sample, fixing the sample, extracting nucleic acid from the sample, assaying the nucleic acid, e.g., to detect the presence of a biological organism, and optionally analyzing the information collected in the assay to identify the biological organism. The “kit” may be extremely portable, such as the size of a briefcase, may be larger. Larger kits can be the size of a steamer trunk, e.g., about 1 to 3 feet wide, about 3 to 8 feet long, and about 1 to 4 feet deep. Even larger kits can be used according to the invention, such as a van or 18 foot truck with the equipment loose, tied down, or releasably attached, or permanently attached, to the movable structure. Preferably, the kits are sufficiently small to readily fit onto an aircraft and a movable ground-based vehicle for ready transport to the site of an epidemic in virtually any location. A mobile medical unit or hospital may contain the kit on board, or may function as the portable enclosure of the kit of the invention.

The term “about,” as used herein, should generally be understood to refer to both numbers in a range of numerals. Moreover, all numerical ranges herein should be understood to include each whole integer within the range.

### EXAMPLES

The invention is further defined by reference to the following illustrative examples that may be used to prepare or administer the compositions of the present invention. These examples are for illustrative purposes only, and are not to be construed as limiting the appended claims.

### Example 1

### A Protocol for Collection and Extraction of Genomic Material in the Field

Oropharyngeal, cloacal, and tracheal swabs of a subject, such as a chicken, would be taken. The swabs would be suspended in 1.5 ml of BHI and extracted with an RNeasy Mini® kit, (manufactured by Qiagen of Valencia, Calif., USA, MagMax Kit, Ambien). The RNA would be eluted in 60 μl of RNase®-free water.

### Example 2

### A Protocol for Identification of Genomic Material

Collection of Clinical Samples and Virology—All original primary specimens (throat swab/nasal washes) and cultured samples used in this study were collected over 7 (99/00, 00/01, 01/02, 02/03, 03/04, 04/05, and 05/06) and 3 (03/04; 04/05, and 05/06) influenza seasons, respectively, under the auspices of the Department of Defense Global Emerging Infectious Surveillance (DoD-GEIS) network. Primary specimens were collected within the first 48-72 hours of symptom onset from patients presenting with a fever >100.5° F. oral and cough or sore throat. Dacron throat swab specimens were submerged in 3.0 ml viral transport media (M4 MicroTest Multi-Microbe Media). Submerged throat swabs and saline nasal wash material (5 ml) were shipped on dry ice to Brooks City Base, San Antonio, Tex. Propagation of influenza viruses from primary specimens was achieved using the centrifugation-enhanced shell-vial culture technique followed by typing using influenza virus A or B specific monoclonal antibodies (Chemicon International, Temecula, Calif.) per manufacturer's recommendations and fluorescent microscopy. (Daum L T, Canas L C, Smith C B, et al.: Genetic and antigenic analysis of the first A/New Calcdonia/20/99-like H1N1 influenza isolates reported in the Americas. Emerg. Infect. Dis. 2002; 8: 408-412). Aliquots (0.25 ml) of primary specimens (before and after culturing) served as source of specimen RNA.
Extraction of RNA: RNA extraction was achieved using the Qiagen M48 automated bead-based extraction robot (Qiagen, Valencia, Calif.) with the MagAttract Virus Mini M48 kit (Qiagen, Valencia, Calif.) per manufacturer's protocols, eluted in 50 μl of Elution Buffer and stored at −80° C. until used.
Genomic Primer/Probe Design: Primer/probe design was based upon sequence data obtained from the Los Alamos National Laboratory and Department of Defense data bases. Type specific (influenza A and B) assays target a highly conserved region of the matrix protein (MP) gene and were designed based on 100 and 50 alignments common to all 16 influenza A virus subtypes and both influenza B lineages (B/Victoria and B/Yamagata), respectively. H1, H3, and H5 influenza A subtype specific assays target conserved regions of the respective hemagglutinins. H1 primer/probe sequence alignment was achieved using 51 geographically diverse strains obtained during the 2005/06 and 2006/07 influenza seasons including the A/New Calcdonia/20/99 vaccine strain. H3 primer/probe sequence alignment was achieved using 140 H3N2 field strains collected between 2004 and 2006. H5 primer/probe sequence design was based upon alignment analysis of 22 H5N1 clinical isolates representing clades 1 and 2 (subclades 1, 2 and 3) viruses. All primers and probes were procured from Applied Biosystems (Foster City, Calif.).
Real-Time RT-PCR Platforms: The laboratory-based Lightcycler 2.0 instrument (Roche Molecular Diagnostics) and its lightweight portable (50 lbs) version, the Ruggedized Advanced Pathogen Identification Device (R.A.P.I.D., Idaho Technologies, Salt Lake City, Utah) are both 32-well capillary, real-time instruments which employ similar components and operational software. The R.A.P.I.D. is configured within a hardened case and can be employed at the point of care.
Real-Time RT-PCR Amplification: Primer and probe sequences are shown in FIG. 5. Primer pair melting points are within 2° C. and anneal/extend at 58-60° C. The respective probes anneal/extend 8-10° C. higher than that of the primers. Thermocycling operates in a rapid, 2-temperature format with annealing and extension (30 seconds) facilitated by the short nature of the respective amplicons. Due to genetic variability in the influenza viral genome, degenerate nucleotides were placed at specific loci.

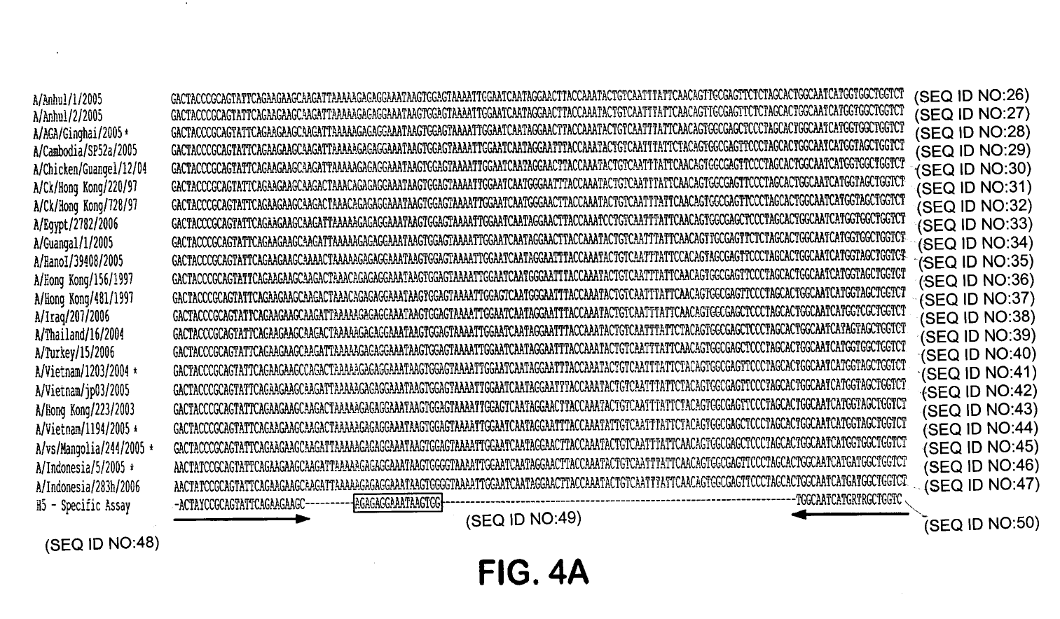

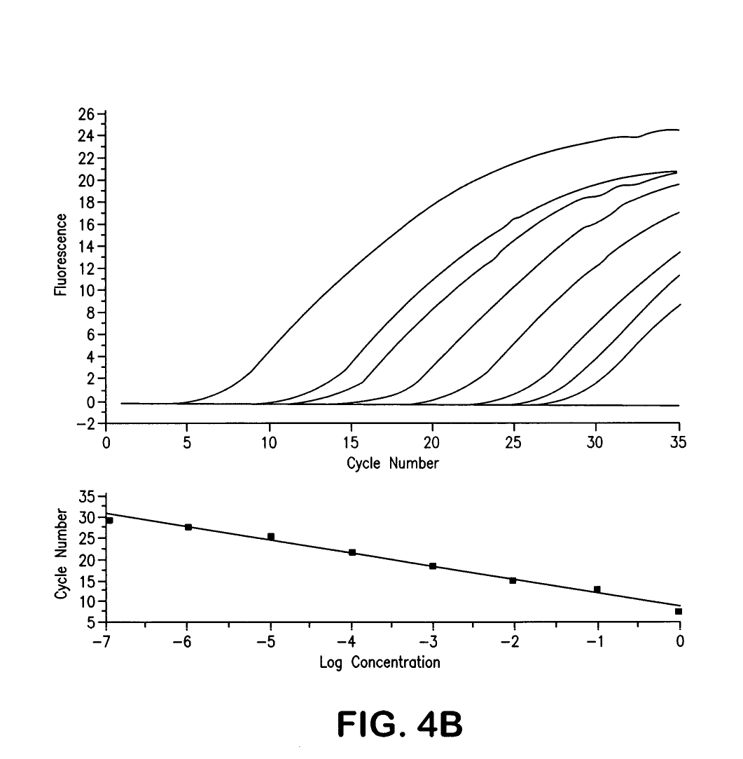

Real-time amplification was performed in a single step, single reaction vessel format. Using the UltraSense Platinum One-Step Quantitative RT-PCR System (Invitrogen, Carlsbad, Calif.), 2 μl RNA was added to 18 μl master mix containing the following components at the indicated final concentrations: IX reaction buffer, 1× enzyme mixture containing 500 nM of each primer and 300 nM probe labeled at the 5′ end with 6-carboxyfluorescein (FAM) reporter dye and at the 3′ end with a nonfluorescent quencher and minor groove binder. Thermocycling was carried out as follows: 30 minutes at 45° C. and 2 minutes at 95° C. for reverse transcription (RT) and denaturation, respectively, followed by 40 amplification cycles consisting of 95° C. for 5 seconds (denaturation) and 60° C. for 30 seconds (extension). Amplification efficiency was determined using the C_{T} slope method (cf. FIG. 4, Frame B) according to the equation: E=[10^{(-1/Slope)}−1]×100. All assays described here exhibited greater than 98.5% amplification efficiency.

For each analysis, ‘no template’ and ‘positive’ controls were included. Baseline fluorescence for each analysis was manually adjusted to that of the respective ‘no template’ control reaction. The ‘positive’ control (0.1 ng cRNA) gives rise to an increase in fluorescence intensity relative to the no template baseline (C_{T} value between 18 and 22). A ‘positive’ unknown is defined as amplification exceeding baseline fluorescence with a corresponding C_{T} value not exceeding 36 in a 40 cycle run. All original, i.e., uncultured specimens and cultured samples reported here using both platforms exhibited C_{T} range values of 26-35 (n=144, mean=31.5) and 17-27 (n=407, mean=23), respectively.

Generation of CRNA Target Templates: Reverse and forward primers for in vitro generation of target complementary RNA (cRNA) templates corresponding to Influenza type (A/B) and Influenza A subtype (H1, H3, and H5) RNA sequences are shown in FIG. 5.

Briefly, traditional RT-PCR was carried out as follows: 5 μl viral RNA was added to a 45 μl master mix containing the following components at the indicated final concentrations: 1× reaction buffer with 1.6 mM MgSO₄, 1× enzyme mixture containing 400 nM HA or MP primer pairs using the SuperScript III One-Step RT-PCR System (Invitrogen, Carlsbad, Calif.). Reverse transcription was carried out at 50° C. for 30 minutes followed by a ‘hot start’ step (2 minutes) at 95° C. Thermocycling (40 amplification cycles) was carried out as follows: 30 seconds at 95° C., 15 seconds at 52° C., 1 minute at 68° C. with final extension for 7 minutes at 68° C. PCR reaction product (5 μl) was subjected to analytical electrophoresis on 2% pre-cast gels containing ethidium bromide (Invitrogen, Carlsbad, Calif.) and remaining product (45 μl) purified using the Qiaquick PCR Purification Kit (Qiagen, Valencia, Calif.). In vitro transcription was carried out for 4 hours using the T7 MegaScript Kit (Ambion, Austin, Tex.) per manufacturer's recommendations. Reactions were subjected to nuclease digestion using Turbo DNase (Ambion, Austin, Tex.) and subsequently purified using the MegaClear kit (Ambion, Austin, Tex.). RNA was quantitated using a NanoDrop (NanoDrop Technologies, Wilmington, Del.) spectrophotometer, aliquoted and served as control cRNA.

Nucleotide Sequencing: Purified amplicons were cloned using a Topo 2.0 Cloning Kit (InVitrogen, Carlsbad, Calif.) and sequenced using the Big Dye Terminator v3.1 reagent Kit. Unincorporated fluorescent nucleotides were removed using a Dye Ex 96-well plate kit per manufacturer's recommendations (Qiagen, Valencia, Calif.). Nucleotide sequencing was performed using an ABI 3100 Genetic Analyzer (ABI Inc., Foster City, Calif.).

### Results:

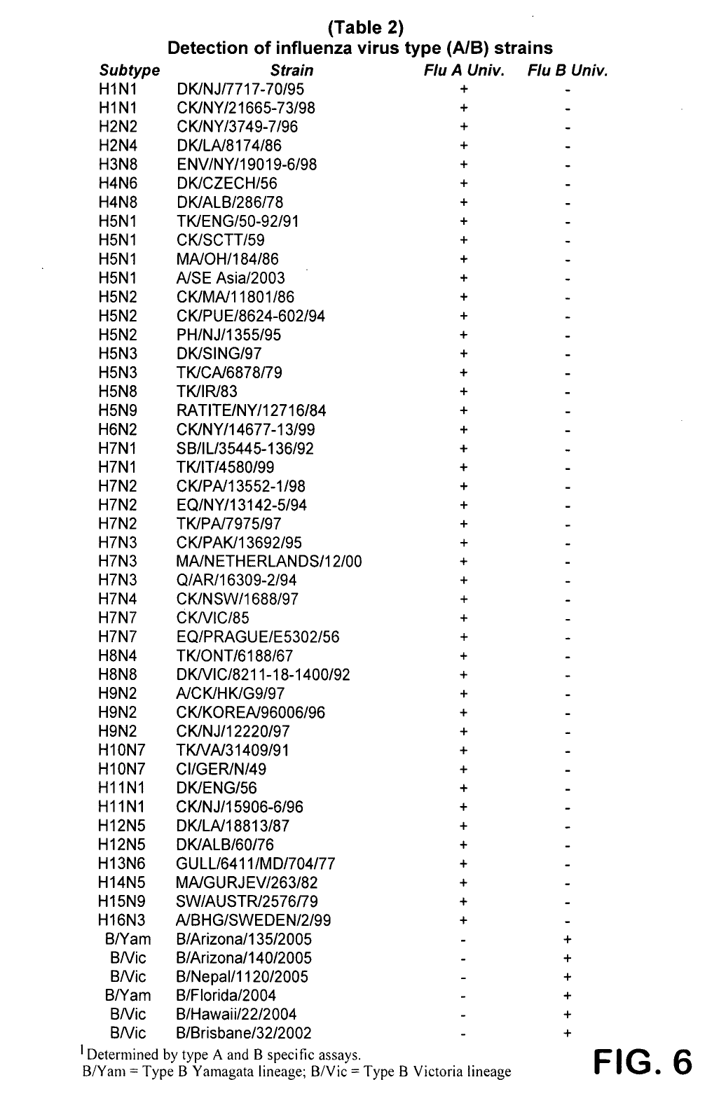

As shown in FIG. 6, influenza A and B virus type specific assays detected all known influenza A hemagglutinin subtypes (H1-H16) and both type B (Yamagata and Victoria) viruses, respectively. Importantly, no cross-reactivity was observed between influenza A and B virus specific probes supporting the very specific nature of these assays. (Total RNA for influenza virus A H1-15 subtypes was obtained from Dr. David Suarez, Southeast Poultry Research Laboratory, USDA Agricultural Research Service, Athens, Ga. 30605. H16 RNA was provided by Drs. R. A. Fouchier and A. D. Osterhaus of the Department of Virology, Erasmus Medical Center, The Netherlands. Influenza B virus reference strains were obtained from the Department of Defense Global Emerging Infectious Surveillance (DoD-GEIS) network.)

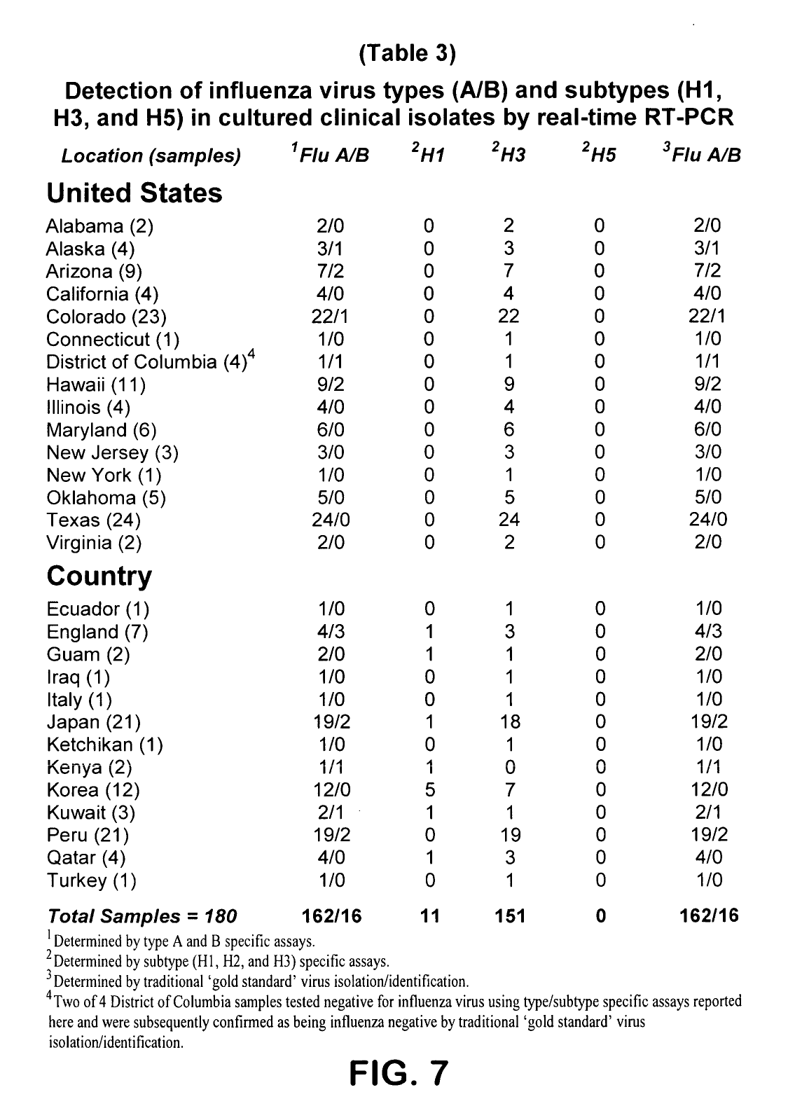

H1, H3, and H5 influenza A subtype specific assays were initially evaluated using 180 archived clinical isolates. As shown in FIG. 7, 178 samples were correctly typed and subtyped in research blinded fashion; the far greater number of samples identified as type A influenza (91%) with the remainder, i.e., 9% being type B influenza. Of the type A influenza samples, the H3 subtype was the most prevalent (93.2%) with the remaining 6.8% H1 subtype. Two samples (District of Columbia) tested influenza virus negative (using type and subtype assays reported here) and were later confirmed as being Coxsackie B and Adenovirus (data not shown). Consistent with no cross-reactivity of type specific probes, no subtype specific probe cross-reactivity, i.e., H1, H3, and H5 assay cross-reactivity was observed. Furthermore, 40 commonly encountered bacteria/viruses were tested concurrently and did not amplify using either type and subtype specific primer/probes listed in Table I (data not shown).

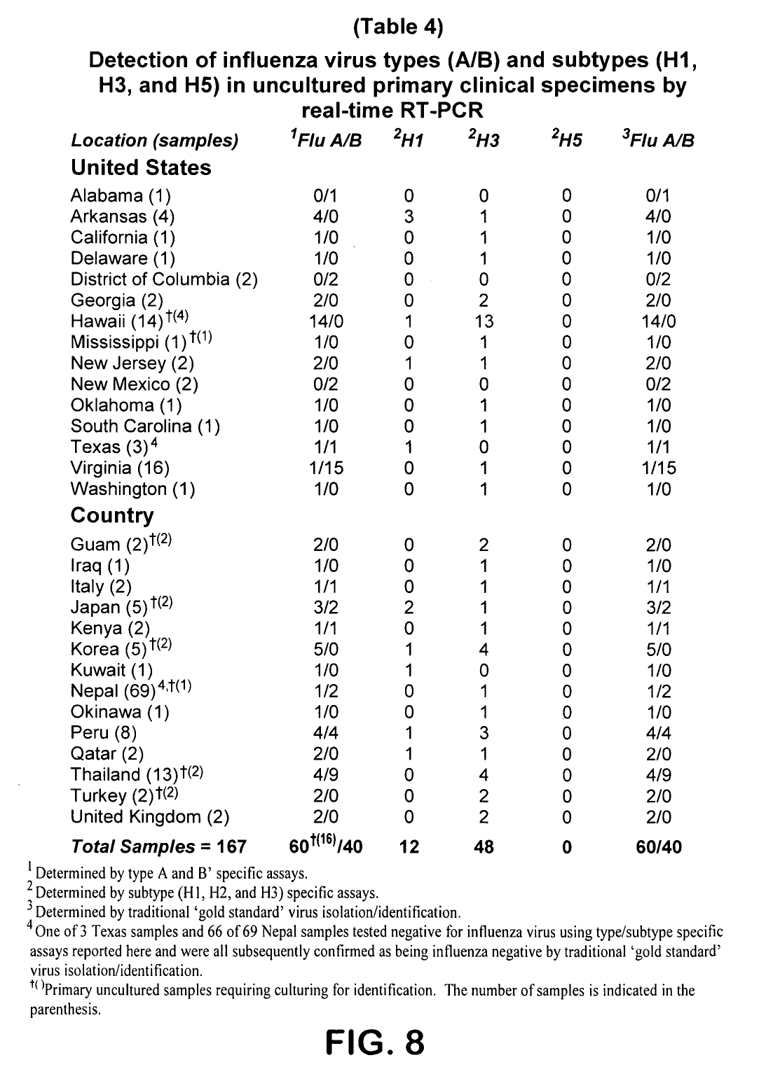

Typing and subtyping influenza viruses at the point of care using uncultured (low viral titer) primary specimens is crucial for expanded surveillance. Shown in FIG. 8 is analysis of 167 uncultured primary clinical samples. Of the 167 specimens, 100 were correctly identified, i.e., typed (Influenza A or B, 60 and 40, respectively) and type A samples subtyped H1 or H3, 12 (20%) and 48 (80%), respectively. Furthermore, 67 negative influenza samples were subsequently determined to be culture negative for influenza viruses. Of the 60 influenza A samples, 16 initially tested influenza negative in contrast to original culture data. The inability to detect 16 of the 100 influenza specimens could have arisen from base pair sequence ‘drift’ in the respective primer/probe binding regions, less than threshold amounts of extracted target RNA arising from prolonged storage/degradation at −80° C. and poor sample collection. Therefore, aliquots of all 16 samples were removed and transferred to monolayer PMK culture tubes and Shell Vials for further analysis. After 48 hours, 10 of the 16 Shell Vial enriched samples tested positive by type specific rRT-PCR and standard immunoreagent fluorescence staining. The remaining 6 samples were checked/screened daily thereafter and 3 of 6 tested positive 5 days post inoculation and the remaining three testing positive 9 days post inoculation. All 16 influenza type A amplicons were further validated by sequence analysis (data not shown). The overall specificity of assays using original specimens as source of RNA was 100% (no cross hybridization) with an overall sensitivity of 90.4% (151 correct positives and negatives out of a total of 167 samples).

Although no H5 influenza virus was observed in our sample collection (FIGS. 7 and 8), the usefulness of the H5 subtype specific assay is demonstrated by alignment analysis of known H5 influenza A virus with H5 primer/probe sequences and H5 assay sensitivity by template serial dilution. Shown in FIG. 1 (Frame A) is alignment analysis of 22 H5 influenza A hemagglutinin sequences including an isolate from the first human H5 outbreak (1997) and subsequent outbreaks through 2006 with the H5 subtype specific, primer/probe sequences. Complete (100%) primer/probe, H5 virus sequence homology (avian and mammalian sources) was observed. Shown in FIG. 1 (Frame B) is a representative profile of serially diluted H5 cRNA template (obtained from a human fatality) over an 8-log dilution range (10⁻⁹ to 10⁻¹⁶ gms) corresponding to approximately 100 H5 cRNA target molecules (10⁻⁶ gms). Serially diluted cRNA targets (types A and B and subtypes H1 and H3) exhibited very similar profiles to that shown in FIG. 1, Frame B (data not shown).

### Conclusions:

Influenza type specific assays described in this report detected all 16 known type A viruses including the recently discovered H16 strain as well as both Yamagata and Victoria type B viruses. Fouchier R A, Munster V, Wallensten A, et al.: Characterization of a novel influenza A virus hemagglutinin subtype (H16) obtained from black-headed gulls. J Virol 2005; 79: 2814-2822. Using both the laboratory-based Lightcycler 2.0 instrument and its lightweight (50 lbs) version, the Ruggedized Advanced Pathogen Identification Device (R.A.P.I.D.), 347 archived primary clinical samples (throat swab/nasal wash, 180 cultured and 167 uncultured), were typed, i.e., influenza A or B and if A, which subtype. Of the 347 total samples evaluated, 278 were correctly identified as being influenza type A (222) or B (56), and all type A were subsequently subtyped as either H1, H3 or H5. Influenza negative (69) samples (Tables 7 and 8: 2 from the District of Columbia, 66 from Nepal and 1 from Texas, respectively) were subsequently confirmed as Coxsackie B, Adenovirus, Parainfluenzas 1, 2, and 3 or virus negative (data not shown). Sixteen of the 100 uncultured primary clinical specimens (FIG. 4) required subsequent culturing. Although real-time RT-PCR is capable of detecting the presence of nucleic acid from nonviable virus, this was not the case since all 16 primary samples were successfully cultured and identified suggesting target RNA degradation from prolonged storage at −80° C., poor sample collection or lower sensitivity in clinical samples than that observed for serially diluted cRNA.

Assay specificity is underscored by the absence of any cross-reactivity, i.e., false positives (of particular note H5). False negatives can arise for a variety of reasons, i.e., presence of RT-PCR inhibitors, low initial viral titer, RNA degradation, poor/low RNA recovery during extraction, user error, or reagent degradation. Although no notable differences in RNA recovery have been observed using manually extracted template compared to robotic extracted template (data not shown), inclusion of an internal positive control could be of value in monitoring the process from extraction through amplification. Das A, et al. Development of an internal positive control for rapid diagnosis of avian influenza virus infections by real-time reverse transcription-PCR with lyophilized reagents. J Clin Microbiol 2006; 44: 3065-3073. It should be understood that this reference provides guidance on suitable lyophilization techniques that can be used in accordance with the present invention, and therefore this references is incorporated herein by express reference thereto.

### Example 3

128 samples from 64 cotton rats were taken from lung and nasal tissue. Some of the animals were influenza virus culture negative. cRNA was extracted from the samples and added to the prime mix. Real time rRT PCR analysis using ABI 7500 was performed, and influenza RNA was detected in the samples within 90 minutes. Importantly, influenza was detected at levels of about 1 to >100 influenza viruses in tissues.

Although preferred embodiments of the invention have been described in the foregoing description, it will be understood that the invention is not limited to the specific embodiments disclosed herein but is capable of numerous modifications by one of ordinary skill in the art. It will be understood that the materials used and the chemical and pharmaceutical details may be slightly different or modified from the descriptions herein without departing from the methods and compositions disclosed and taught by the present invention.

1. A biological organism identification product that comprises:
a collection device to collect one or more sample organisms;
a fixing and transporting composition present in an amount sufficient to kill the one or more sample organisms associated with the collection device;
an extraction member to extract a sufficient amount of genomic nucleic acid from the one or more sample organisms to facilitate identification thereof; and
a substantially stable polymerase chain reaction (PCR) component into which the sufficient amount of genomic nucleic acid can be added.

2. The product of claim 1 further comprising:
a portable enclosure to retain the product components comprising the collection device, fixing and transporting composition, extraction member, and stabilized component.

3. The product of claim 2, wherein the portable enclosure further comprises a polymerase chain reaction machine.

4. The product of claim 3, wherein the product further comprises a plurality of active pharmaceutical ingredient doses in an amount sufficient to prevent or treat one or more conditions caused by the identified biological organism.

5. The product of claim 1, wherein the PCR component is lyophilized to resist temperatures of about 30° C. for at least about 8 hours.

6. The product of claim 3, wherein the fixing and transporting composition comprises alcohol, sodium cyothianate, or combination thereof.

7. The product of claim 3, wherein the polymerase chain reaction component includes a plurality of premixed polymerase chain reaction reagents in one or more vessels.

8. The product of claim 7, wherein the vessels are adapted and configured to operate in connection with a desired polymerase chain reaction device.

9. The product of claim 7, wherein the stabilized component comprises one or more primers specific to the detection of one or more predetermined biological organisms.

10. The product of claim 9, wherein the biological organism includes a bacteria, virus, or parasite.

11. The product of claim 10, wherein the virus is influenza.

12. The product of claim 11, wherein the influenza is type A, B, or a combination thereof and the identification is specific to the influenza type.

13. A method of identifying a biological organism that comprises:
collecting a biological sample from a subject;
fixing the biological sample in a sufficient amount of a fixing agent to minimize or eliminate any contamination by the biological sample;
extracting a sufficient amount of genomic nucleic acid from the fixed biological sample;
assaying the sufficient amount of the genomic nucleic acid in a substantially stable polymerase chain reaction component to obtain information about the organism, wherein the polymerase chain reaction component further comprises a sufficient amount of one or more primers each of which is chemically associable to a protein component specific to a biological organism; and
analyzing the information to identify the biological organism, wherein no more than about 24 hours passes from the collecting to the assaying to obtain information.

14. The method of claim 13, wherein the assaying comprises dissolving the genomic nucleic acid in one or more polymerase chain reaction components.

15. The method of claim 13, wherein the assaying is conducted for about 60 to 150 minutes.

16. The method of claim 13, wherein the polymerase chain reaction component is prepared by premixing a plurality of polymerase chain reaction reagents.

17. The method of claim 20, wherein the analyzing is completed within 30 minutes to 3 hours of collecting the sample.

18. A reagent mixture for detection of a microbial sequence, the reagent-mixture comprising one or more microbe-specific primers, probes, or enzymes, or a combination thereof, present in a mixture that is at least substantially stable at room temperature and is adapted and configured for use with a polymerase chain reaction (PCR) device.

19. The reagent mixture of claim 18, wherein the further mixture is substantially stable at room temperature for at least about five days up to about two weeks.

20. The reagent mixture of claim 18, wherein the detection of the microbial sequence occurs within about 90 minutes after the microbial sequence is extracted from a sample.

21. The reagent mixture of claim 19, wherein the microbial sequence is from an influenza virus.

22. The reagent mixture of claim 21, wherein the reagent mixture comprises an influenza strain A probe with the sequence (FAM)-tcaggccccctcaaagc, an influenza strain B probe with the sequence (FAM)-atgggaaattcagctct, an influenza subtype H1 probe with the sequence (FAM)-tctccaaagtatgtcagg, an influenza subtype H3 probe with the sequence (FAM)-tgagatcagatgcacccat, an influenza subtype H5 probe with the sequence (FAM)-agagrggaaataagtgg, or any combination thereof.

23. The reagent mixture of claim 18, wherein the reagent mixture is configured and adapted used to identify one or more sample biological organisms that have been collected by a collection device.

24. The reagent mixture of claim 23, wherein the further mixture is used to identify the one or more samples at a field site or a remote location.

25. The reagent mixture of claim 18, wherein the reagent mixture is contained in a liquid form.

26. The reagent mixture of claim 18, wherein the reagent mixture is present in a liquid form in a test tube, a 96-well plate, or a capillary vessel.

27. The reagent mixture of claim 18, wherein the mixture is lyophilized.

28. A method for detecting a microbial sequence which comprises:
obtaining genomic nucleic acid from a biological sample; and
assaying the genomic nucleic acid by adding the nucleic acid to a mix which comprises one or more microbe-specific primers, probes, enzymes or combinations thereof,
wherein the mix is at least substantially stable at room temperature and is configured and adapted for use with a polymerase chain reaction (PCR) device.

29. The method of claim 28, wherein the assaying further comprises adding the mix to the polymerase chain reaction (PCR) device, running the assay, and completing the assay in less than about 90 minutes.

30. The method of claim 29, wherein the assaying further comprises detecting the microbial sequence in real-time using fluorescence equipment adapted for use with the PCR device.

31. The method of claim 28, wherein the genomic material is from a bacteria or virus.

32. The method of claim 28, wherein the genomic material is from an influenza virus.

33. The method of claim 31, wherein the mixture comprises an influenza strain A probe with the sequence (FAM)-tcaggccccctcaaagc, an influenza strain B probe with the sequence (FAM)-atgggaaattcagctct, an influenza subtype H1 probe with the sequence (FAM)-tctccaaagtatgtcagg, an influenza subtype H3 probe with the sequence (FAM)-tgagatcagatgcacccat, an influenza subtype H5 probe with the sequence (FAM)-agagrggaaataagtgg, or any combination thereof.
